(19) United States
(12) Patent Application Publication
(10) Pub. No.: US 2012/0329842 A1
(43) Pub. Date: Dec. 27, 2012

(54) SMALL MOLECULE COMPOUNDS AS BROAD-SPECTRUM INHIBITORS OF METALLO-BETA-LACTAMASES

(76) Inventors: Yongcheng Song, Pearland, TX (US); Timothy Palzkill, Houston, TX (US); Pinhong Chen, Houston, TX (US); Lori Horton, Katy, TX (US)

(21) Appl. No.: 13/332,793

(22) Filed: Dec. 21, 2011

Related U.S. Application Data
(60) Provisional application No. 61/427,010, filed on Dec. 23, 2010.

(51) Int. Cl. A61K 31/426; C07D 277/56; A61P 31/04; C12N 9/99

(52) U.S. Cl. 514/365; 435/184; 548/201

(57) ABSTRACT

The present invention concerns methods and/or compositions for treatment and/or prevention of bacterial infection wherein the bacteria has at least one metallo-β-lactamase. The bacteria are provided with an inhibitor of the metallo-β-lactamase, for example in conjunction with an antibiotic that targets the bacteria. The bacteria may be a drug-resistant strain or susceptible to becoming a drug-resistant strain. In specific embodiments, the bacteria is Pseudomonas or Acinetobacter spp.

### CROSS-REFERENCE TO RELATED APPLICATIONS

This application claims priority to U.S. Provisional Patent Application Ser. No. 61/427,010, filed on Dec. 23, 2010, which is incorporated by reference herein in its entirety.

### STATEMENT REGARDING FEDERALLY SPONSORED RESEARCH OR DEVELOPMENT

This invention was made with government support under Grant No. R21AI090190, awarded by the National Institutes of Health. The government has certain rights in the invention.

### TECHNICAL FIELD

The present invention concerns at least the fields of cell biology, molecular biology, medicinal chemistry, medicine, and bacteriology. In specific aspects of the invention, the present invention concerns antibiotic therapy.

### BACKGROUND OF THE INVENTION

β-Lactams are widely prescribed antibiotics to treat bacterial infections with the introduction of the first drug in the class, penicillin G, dating back to the 1940s. They consist of six different structural subtypes including penams, cephems, monobactams, penems, carbapenems, and clavams (FIG. 1). Penams, such as penicillin G and methicillin, are the prototype of this class of antibiotics. They contain a core structure of a bicyclic ring consisting of a 4-membered β-lactam and a 5-membered tetrahydrothiazole. Cephems, including cephalosporins (e.g., cephaloridine and nitrocefin) and cephamycins, have a core structure of a β-lactam ring fused with a 6 membered dihydro-2H-thiazine ring. In monobactams, such as aztreonam, the β-lactam ring is not fused with another ring. Penems (e.g., faropenem) and carbapenems (e.g., imipenem), differ from penams by the presence of a double bond in the five-membered ring. Clavulanic acid, which is a representative of the clavams, does not exhibit antibacterial activity but is an inhibitor of many active site serine β-lactamases.

The development of β-lactam antibiotics over the past 60 years has lead to the availability of drugs to treat a wide range of Gram-positive and Gram-negative bacterial infections. However, during the past few decades, populations of drug resistant bacteria have increased significantly. For example, methicillin-resistant staphylococcal infections have increased from 2% in 1980 to >60% at present and vancomycin-resistant enterococcal infections have increased from 0% to >20% in the same period (13). Moreover, Gram-negative bacterial infections have now become significant clinical challenges, especially in the cases of P. aeruginosa and Acinetobacter spp (14-18). Pseudomonas infections alone account for ˜10 percent of hospital-acquired infections. This Gram-negative bacterium is well known for its acquired resistance to antibiotics and is therefore a particularly troublesome pathogen. Only a few antibiotics are effective for treatment, including imipenem and fluoroquinolones, and even these antibiotics are not effective against all strains. New strains resistant to these antibiotics have continued to emerge. In addition, Pseudomonas infections are life-threatening for patients with cystic fibrosis and severe burns, as well as for immuno-compromised cancer and AIDS patients.

One important mechanism bacteria use to resist β-lactam antibiotics is the production of β-lactamases, which can hydrolyze the 4-membered β-lactam ring and render the drugs inactive. There are two mechanistically distinct types of β-lactamases (19): serine β-lactamases and metallo-β-lactamases (MBLs). The class A, C and D β-lactamases belong to the first type and use a serine —OH group as a nucleophile to attack and hydrolyze the amide bond in the β-lactam ring. In contrast, the class B β-lactamases are Zn²⁺ dependent metalloenzymes, with one or two Zn ions at the active site. The generally accepted mechanism for MBLs is that the coordination of the carbonyl group of the β-lactam to the metal ion(s) will polarize and thereby activate it. The nucleophilic attack by a nearby hydroxide/water ligand on Zn²⁺ will then result in the hydrolysis of the β-lactam ring (20, 21). The serine β-lactamases have been studied extensively during the past few decades and several potent, broad-spectrum inhibitors have been identified and developed, among which, clavulanic acid, sulbactam and tazobactam, combined with various β-lactam antibiotics, have been used clinically to combat otherwise β-lactam-resistant bacterial infections (22).

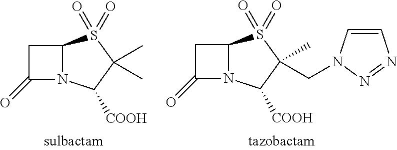

Although the first metallo-β-lactamase was described in 1966 (23), the clinical significance of these enzymes has only recently been recognized. The importance of these enzymes came to light when plasmid-encoded, transferable MBLs were found to spread quickly among many species of Gram-negative bacteria around the world and confer resistance to imipenem and extended-spectrum cephalosporins (2, 3, 5). For example, the IMP-1 enzyme was first identified in a strain of P. aeruginosa in Japan in 1988 (6), and it has now been found in at least 18 Gram negative pathogenic species in many counties (9, 24-28). Another widely disseminated and clinically significant type of transferable MBL genes are VIMs (VIM-1 to VIM-11) (29-34). Infections caused by Gram-negative bacteria bearing these MBLs have been found to be a significant challenge in the clinic.

The most worrisome feature of metallo-β-lactamases is that they are able to hydrolyze essentially all β-lactams, including carbapenems such as imipenem, which have the widest antibacterial spectrum and are one of the very few effective drugs to treat some Gram-negative bacterial infections, such as P. aeruginosa. Only monobactams, such as aztreonam (FIG. 1), are not substrates of MBLs (31, 35). However, clinical applications of aztreonam are rather limited. There is, therefore, a pressing need to find potent, drug-like inhibitors that are active against a wide spectrum of MBLs, especially clinically relevant plasmid-encoded MBLs (subclass B1, described below).

There has been much interest in discovering and developing MBL inhibitors both from academia and the pharmaceutical industry during the past decade (2-4). A number of structurally diverse inhibitors have been identified, such as thiol-containing compounds (36-39), trifluoromethyl alcohols and ketones (40), tetrazoles (11) and succinates (12). Several representative inhibitors are shown in FIG. 2. However, these compounds are not good drug candidates for further development due to one or more of the following reasons: 1) chemical instability (e.g., thiols); 2) a narrow spectrum of activity or weak activity; and 3) side effects (e.g., captopril, a potent inhibitor of angiotensin-converting enzyme, used to lower blood pressure to treat hypertension).

Despite limited success in developing clinically useful MBL inhibitors, structural biological studies using these compounds have revealed useful information for elucidation of enzyme mechanism and future drug design. In addition, due to the low homology among MBLs from different species, the 3-D structures of these enzymes also facilitated their classification (3, 20). Subclass B1 is the largest MBL family, consisting of the majority of enzymes and including clinically important transferable enzymes such as the IMPs and VIMs. The B1 enzymes feature a conserved Zn(II) binding motif with the sequence HXHXD. There are two Zn²⁺ at the active site of B1 enzymes, with Zn1 bound by three His residues and Zn2 by one His, one Cys and one Asp, as shown in FIG. 3A. In addition, there is a bridging water ligand, or most likely a hydroxide, between the two Zn ions, which is believed to be the nucleophile that hydrolyzes the bound substrate. Moreover, a unique feature of this subclass metallo-β-lactamases with respect to serine β-lactamases is that there is a flexible loop, which is a part of the protein active site (3, 20). Crystallographic (11, 41) and NMR (42) studies show that the conformation of this loop is very dynamic: it, like a cap, opens and closes when a substrate or an inhibitor moves into the enzyme. It is mainly hydrophobic in nature and enhances substrate/inhibitor binding. The flexibility provided by the loop enables the enzyme to accommodate and then hydrolyze a wide range of β-lactams. The B2 MBLs only bind one Zn ion that corresponds to the Zn2 for B1 enzymes, as one of the His residues that coordinates to Zn1 is replaced by an Asn in B2 class enzymes, as shown in FIG. 3B. The B3 family MBLs can also bind two Zn ions, where, compared to B1 enzymes, the Cys residue coordinating to Zn2 is replaced by a Ser residue (FIG. 3C).

It is apparent that a pressing need exists to design and develop novel inhibitors that have broad and potent activity against MBLs. The successful development of such inhibitors would offer new treatment options for epidemic drug resistant Gram-negative bacterial infections. The present invention provides for such a need.

### BRIEF SUMMARY OF THE INVENTION

The present invention is directed to methods and compositions for prophylaxis and/or treatment of an individual for pathogenic infection, including bacterial infection or susceptibility to bacterial infection. The bacteria may be Gram positive or Gram negative. In particular embodiments, the bacteria comprises metallo-β-lactamases (MBL). In specific embodiments, the MBL acts to render ineffective an antibiotic of choice such that the bacteria is resistant to the antibiotic of choice; the present invention provides one or more MBL inhibitors that inhibit activity of the MBL such that the antibiotic of choice is thereby effective against the target bacteria. In specific cases, the MBL inhibitors render bacteria, including at least the majority of a certain pathogenic bacterial population in an individual, for example, sensitive to a therapy that otherwise the bacteria would be resistant to; in particular aspects such bacteria produce one or more MBLs.

In some cases, a bacterial population to which an individual is exposed or that is harbored by an individual is sensitive to a particular beta-lactam antibiotic but, over a period of time, part or all of the majority of the population becomes resistant to the particular beta-lactam antibiotic. In specific embodiments, the bacteria become resistant by producing metallo-beta-lactamases that inhibit the particular beta-lactam antibiotic. The MBL inhibitors of the invention either directly or indirectly inhibit the activity of the MBLs, although in alternative embodiments the MBL inhibitors of the invention act through another mechanism to render a particular antibiotic effective against a certain bacteria.

In specific embodiments of the invention, one or more inhibitors that block one or more metallo-beta-lactamases are delivered to an individual in need thereof, which in specific embodiments is an individual that has one or more symptoms of a bacterial infection or is at risk for developing a bacterial infection, for example. In specific embodiments, the one or more inhibitors are used in combination with a beta-lactam antibiotic. The MBL inhibitor and the beta-lactam antibiotic may be formulated separately or together, such as in a single pill, tablet, liquid, aerosol or cream, for example.

In some embodiments of the present invention, there are methods and/or compositions for fighting bacteria that can develop or have developed resistance to β-lactam antibiotics, for example by synthesizing a β-lactamase that attacks the β-lactam ring. Although one can employ inhibitors for serine β-lactamases in conjunction with the antibiotic of choice, such inhibitors are ineffective against metallo-β-lactamases. Therefore, the present invention provides inhibitors that are effective against metallo-β-lactamases and that may be used in conjunction with an antibiotic that targets a bacteria that comprises at least one metallo-β-lactamase.

In some embodiments of the invention there is use rational, structure based approaches to design and synthesize novel, broad-spectrum metallo-β-lactamase inhibitors, for example targeting the clinically important class B1 enzymes. X-ray crystallography can also be used to study the interactions between the enzymes and inhibitors, for example in an effort to facilitate the design of compounds with improved activity.

In some embodiments, there is a composition comprising a compound selected from the group consisting of (R)-4,5-dihydro-2-phenylthiazole-4-carboxylic acid; 2-benzylthiazole-4-carboxylic acid; N-benzyl-6-hydroxypyridine-2-carboxamide; 1-hydroxy-5-phenylpyridin-2-one; (R)-2-(2-hydroxyphenyl)thiazolidine-4-carboxylic acid; 2-[3-(tert-butoxycarbonylamino)phenyl]-4,5-dihydrothiazole-4-carboxylic acid; 2-(3-aminophenyl)-4,5-dihydrothiazole-4-carboxylic acid; (S)-2-(4-bromophenyl)-4,5-dihydro-5,5-dimethylthiazole-4-carboxylic acid; (S)-2-(3-acetamidophenyl)-4,5-dihydro-5,5-dimethyolthiazole-4-carboxylic acid; tert-butyl ((R)-4-(methoxycarbonyl)-4,5-dihydrothiazol-2-yl)methylcarbamate, and a mixture thereof. The composition may be further defined as a variant of a compound selected from the group consisting of (R)-4,5-dihydro-2-phenylthiazole-4-carboxylic acid; 2-benzylthiazole-4-carboxylic acid; N-benzyl-6-hydroxypyridine-2-carboxamide; 1-hydroxy-5-phenylpyridin-2-one; (R)-2-(2-hydroxyphenyl)thiazolidine-4-carboxylic acid; 2-[3-(tert-butoxycarbonylamino)phenyl]-4,5-dihydrothiazole-4-carboxylic acid; 2-(3-aminophenyl)-4,5-dihydrothiazole-4-carboxylic acid; (S)-2-(4-bromophenyl)-4,5-dihydro-5,5-dimethylthiazole-4-carboxylic acid; (S)-2-(3-acetamidophenyl)-4,5-dihydro-5,5-dimethyolthiazole-4-carboxylic acid; tert-butyl ((R)-4-(methoxycarbonyl)-4,5-dihydrothiazol-2-yl)methylcarbamate, and a mixture thereof. In at least specific cases, the compound restores the antibacterial activity of a β-lactam antibiotic. Variants of these compounds are encompassed in the invention. Such variants may have one or more different R groups or changes compared to these compounds, and such variations may be standard alterations; exemplary changes are provided in the Definitions section herein, merely as examples, however.

In some embodiments, there is a method of treating a bacterial infection in an individual, comprising the step of administering a therapeutically effective amount of a composition of the invention to the individual. In specific cases, the bacterial infection is a drug-resistant bacterial strain. The individual may or may not exhibit one or more symptoms of the bacterial infection. The bacterial infection may or may not be definitively identified as a resistant bacteria prior to treatment.

In some embodiments, there is a pharmaceutical composition comprising a compound selected from the group consisting of compound (R)-4,5-dihydro-2-phenylthiazole-4-carboxylic acid; 2-benzylthiazole-4-carboxylic acid; N-benzyl-6-hydroxypyridine-2-carboxamide; 1-hydroxy-5-phenylpyridin-2-one; (R)-2-(2-hydroxyphenyl)thiazolidine-4-carboxylic acid; 2-[3-(tert-butoxycarbonylamino)phenyl]-4,5-dihydrothiazole-4-carboxylic acid; 2-(3-aminophenyl)-4,5-dihydrothiazole-4-carboxylic acid; (S)-2-(4-bromophenyl)-4,5-dihydro-5,5-dimethylthiazole-4-carboxylic acid; (S)-2-(3-acetamidophenyl)-4,5-dihydro-5,5-dimethyolthiazole-4-carboxylic acid; tert-butyl ((R)-4-(methoxycarbonyl)-4,5-dihydrothiazol-2-yl)methylcarbamate, and a mixture thereof; the pharmaceutical composition comprises a pharmaceutically acceptable carrier.

In some embodiments, the composition is further defined as a variant of a compound selected from the group consisting of (R)-4,5-dihydro-2-phenylthiazole-4-carboxylic acid; 2-benzylthiazole-4-carboxylic acid; N-benzyl-6-hydroxypyridine-2-carboxamide; 1-hydroxy-5-phenylpyridin-2-one; (R)-2-(2-hydroxyphenyl)thiazolidine-4-carboxylic acid; 2-[3-(tert-butoxycarbonylamino)phenyl]-4,5-dihydrothiazole-4-carboxylic acid; 2-(3-aminophenyl)-4,5-dihydrothiazole-4-carboxylic acid; (S)-2-(4-bromophenyl)-4,5-dihydro-5,5-dimethylthiazole-4-carboxylic acid; (S)-2-(3-acetamidophenyl)-4,5-dihydro-5,5-dimethyolthiazole-4-carboxylic acid; tert-butyl ((R)-4-(methoxycarbonyl)-4,5-dihydrothiazol-2-yl)methylcarbamate, and a mixture thereof. In some cases, the variant restores the antibacterial activity of a β-lactam antibiotic.

In some embodiments, there is a method of treating a bacterial infection in an individual, comprising the step of administering a therapeutically effective amount of a composition of the present invention. In specific embodiments, the bacterial infection is a drug-resistant bacterial strain. The bacterial infection is pathogenic, in particular embodiments.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows six exemplary types of B-lactams with core structures illustrated.

FIG. 2 provides selected MBL inhibitors with Zn binding groups illustrated.

FIG. 3 shows MBL active sites of A) B. cereus BCII (subclass B1), B) A. hydrophila CphA (subclass B2), and C)S. maltophilia L1 (subclass B3). Zinc ions are shown as gray spheres, water as red spheres. Figure from ref. 20.

FIG. 4 shows dose responsive curves of compounds 1-4, together with that of captopril.

FIG. 5. (A) Structural similarities of compounds 1 and 2 to β-lactams as well as their hydrolyzed products. (B) The active site of the crystal structure of S. maltophilia L1 MBL complexed with the hydrolyzed product of moxalactam, showing chelation of Zn2 ion by the imino and the carboxylate groups. Zinc ions are shown as gray spheres and water as a red sphere. Figure from ref. 20.

FIG. 6. General structure of thiazolidine compound library and several examples.

FIG. 7. Tautomerization of compounds 3 and 4.

FIG. 8 provides illustration of a variety of MBL inhibitors.

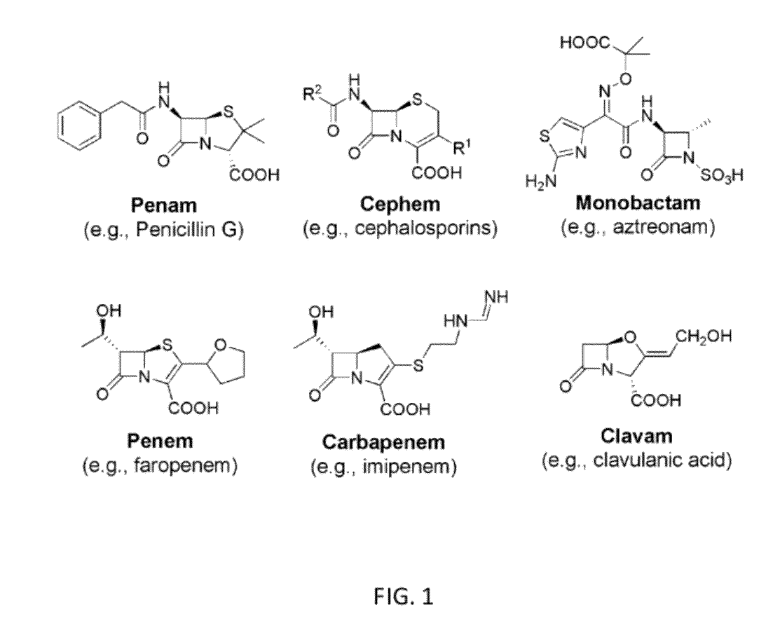

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more elements or steps of the invention, for example. Some embodiments of the invention may consist of or consist essentially of one or more elements, method steps, and/or methods of the invention. It is contemplated that any method or composition described herein can be implemented with respect to any other method or composition described herein.

### I. EXEMPLARY DEFINITIONS

When used in the context of a chemical group, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylimino); “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); and “sulfinyl” means —S(O)— (see below for definitions of groups containing the term sulfinyl, e.g., alkylsulfinyl).

In the context of chemical formulas, the symbol “—” means a single bond, “═” means a double bond, and “≡” means triple bond. The symbol “-” represents an optional bond, which if present is either single or double. The symbol “ [symbol] ” represents a single bond or a double bond. Thus, for example, the structure

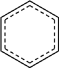

includes the structures

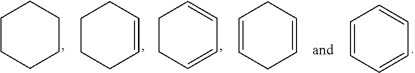

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

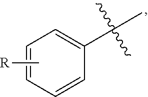

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

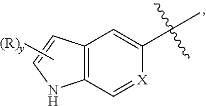

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

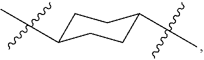

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

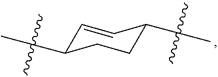

are non-limiting examples of alkenediyl groups. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. The term “alkynediyl” when used without the “substituted” modifier refers to a divalent unsaturated aliphatic group, with two carbon atoms as points of attachment, a linear or branched, cyclo, cyclic or acyclic structure, at least one carbon-carbon triple bond, and no atoms other than carbon and hydrogen. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄—CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

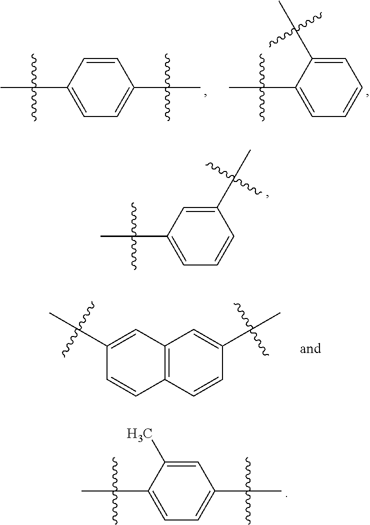

When the term “aryl” is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the group consists of no atoms other than carbon, hydrogen, aromatic nitrogen, aromatic oxygen and aromatic sulfur. As used herein, the term does not preclude the presence of one or more alkyl group (carbon number limitation permitting) attached to the aromatic ring or any additional aromatic ring present. Non-limiting examples of heteroaryl groups include furanyl, imidazolyl, indolyl, indazolyl (Im), methylpyridyl, oxazolyl, pyridyl, pyrrolyl, pyrimidyl, pyrazinyl, quinolyl, quinazolyl, quinoxalinyl, thienyl, and triazinyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of heteroarenediyl groups include:

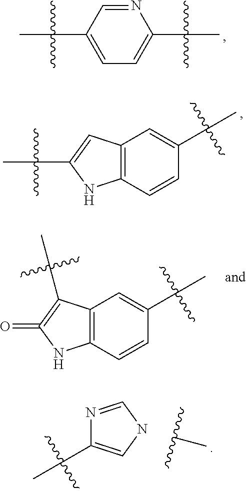

When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄—CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. Similarly, the term “alkylthio” when used without the “substituted” modifier refers to the group —SR, in which R is an alkyl, as that term is defined above. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃. The groups —NHC(O)OCH₃ and —NHC(O)NHCH₃ are non-limiting examples of substituted amido groups.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “arylsulfonyl”, “aralkylsulfonyl”, and “heteroarylsulfonyl”, are defined in an analogous manner. When the term is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —B(OH)₂, —P(O)(OCH₃)₂ or —OC(O)CH₃.

The term “heterocyclic” or “heterocycle” when used without the “substituted” modifier signifies that the compound/group so modified comprising at least one ring in which at least one ring atom is an element other than carbon. Examples of the non-carbon ring atoms include but are not limited to nitrogen, oxygen, sulfur, boron, phosphorus, arsenic, antimony, germanium, bismuth, silicon and/or tin. Examples of heterocyclic structures include but are not limited to aziridine, azirine, oxirane, epoxide, oxirene, thiirane, episulfides, thiirene, diazirine, oxaziridine, dioxirane, azetidine, azete, oxetane, oxete, thietane, thiete, diazetidine, dioxetane, dioxete, dithietane, dithiete, pyrrolidine, pyrrole, oxolane, furane, thiolane, thiophene, borolane, borole, phospholane, phosphole, arsolane, arsole, stibolane, stibole, bismolane, bismole, silolane, silole, stannolane, stannole, imidazolidine, imidazole, pyrazolidine, pyrazole, imidazoline, pyrazoline, oxazolidine, oxazole, oxazoline, isoxazolidine, isoxazole, thiazolidine, thiazole, thiazoline, isothiazolidine, isothiazole, dioxolane, thithiolane, triazole, furazan, oxadiazole, thiadiazole, dithiazole, tetrazole, piperidine, pyridine, oxane, pyran, thiane, thiopyran, salinane, saline, germinane, germine, stanninane, stannine, borinane, borinine, phosphinane, phosphinine, arsinane, arsinine, piperazine, diazine, morpholine, oxazine, thiomorpholine, thiazine, dioxane, dioxine, dithiane, dithiine, triazine, trioxane, tetrazine, azepane, azepine, oxepane, oxepine, thiepane, thiepine, homopiperazine, diazepine, thiazepine, ozocane, azocine, oxecane, or thiocane. When the term “heterocyclic” is used with the “substituted” modifier one or more hydrogen atom has been independently replaced by one of the following exemplary non-limiting functional groups: —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂ or —OC(O)CH₃.

As used herein, a “chiral auxiliary” refers to a removable chiral group that is capable of influencing the stereoselectivity of a reaction. Persons of skill in the art are familiar with such compounds, and many are commercially available.

An “isomer” of a first compound is a separate compound in which each molecule contains the same constituent atoms as the first compound, but where the configuration of those atoms in three dimensions differs.

As used herein, the term “individual” or “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, trifluoroacetic acid, trifluormethyl sulfonic (triflic) acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include, but are not limited to ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient that may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease. In specific embodiments, the onset of a bacterial population becoming resistant to antibiotics is prevented or occurs to a lesser degree as as a result of use of compounds and/or methods the invention.

“Effective amount,” “Therapeutically effective amount” or “pharmaceutically effective amount” means that amount which, when administered to a subject or patient for treating a disease, is sufficient to effect such treatment for the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. GENERAL EMBODIMENTS OF THE INVENTION

In embodiments of the invention, there are compounds employed to treat a bacterial infection, prevent a bacterial infection, or reduce the risk of or overcome the development of resistance to an antibiotic. In specific embodiments, the compound comprises one or more inhibitors that inhibit the activity of one or more metallo-β-lactamases. The invention may be employed for any male or female mammal, including humans, dogs, cats, horses, cows and so forth. In some embodiments, the individual has one or more symptoms of bacterial infection or is at risk for developing infection, such as an individual that has a viral infection, is undergoing a medical or dental procedure, or whose system has been exposed to one or more conditions conducive to bacterial infection, such as incurring a wound. Urinary tract infections, nosocomial infections including pneumonia, infections associated with medical devices, including catheters, are exemplary infections encompassed in the invention.

In some cases the individual has experienced recurrent pathogenic infections. In some cases the individual has had a bacterial infection diagnosed, whereas in other cases the individual has not yet had a bacterial infection diagnosed. The bacterial infection may be diagnosed by sputum test; blood test, including white blood cell count and/or blood culture; test for antibodies; polymerase chain reaction (including RT-PCR), or a combination thereof, for example. There are published procedures in the art for detection of bacteria comprising a metallo-beta-lactamase using microbiology methods with a beta-lactam, with and without EDTA, for example. Samples for bacterial analysis may be collected by known methods in the art, including from pus, mucus, sputum, blood, nasal swab, vaginal swab, urine, feces, and so forth, for example.

The individual may be elderly (65 years of age or older) or an infant, in certain embodiments. The individual may or may not have one or more symptoms of a bacterial infection, such symptom(s) including fever, inflammation, heat, swelling, pain, pus production, or a combination thereof, for example. The infection may be in any body part(s), including the blood, lungs, skin, ear, eye, throat, urinary tract, nose, sex organ, stomach, bowel, and so forth.

In some embodiments, the present invention concerns design and synthesis of novel small molecule compounds that serve as broad-spectrum inhibitors of metallo-β-lactamases (MBLs). This is achieved by using a combination of rational drug design, synthetic chemistry, biological activity testing and x-ray crystallography, for example. The inhibitors are clinically useful drugs that can restore the antibacterial activity of β-lactam antibiotics (e.g., imipenem) against many highly drug resistant bacterial strains, for example.

The development of antibiotics is considered to be one of the most successful stories in drug discovery. However, according to the World Health Organization, bacterial infections are still the number one cause of human deaths, killing ˜6 million people each year worldwide (1). Even in developed countries, such as the U.S., bacterial infections are once again recognized as a significant threat to public health because of widespread, acquired drug resistance. β-Lactam antibiotics such as penicillins and cephalosporins are among the most often used antimicrobial agents. The most prevalent mechanism of bacterial resistance to β-lactam antibiotics is the production of β-lactamases that are able to hydrolyze and thereby inactivate the drugs. Although mechanism-based inhibitors such as clavulanic acid and sulbactam are available for inactivation of active site serine β-lactamases, no inhibitors are available that are broadly active against the many distinct metallo-β-lactamases (2-4). MBLs have now been recognized as an emerging clinical threat in that these enzymes, unlike active site serine β-lactamases, are able to hydrolyze essentially all β-lactams, including carbapenems (e.g., imipenem) which are last resort drugs for several multidrug resistant Gram-negative bacterial (e.g., Pseudomonas aeruginosa and Acinetobacter spp.) infections. In addition, many MBLs (e.g., IMPs and VIMs) are encoded by transferable metallo-β-lactamase genes on plasmids that have disseminated quickly worldwide (2, 3, 5). Some multidrug resistant strains of Pseudomonas and Acinetobacter spp. have already demonstrated significant resistance against imipenem due to MBL genes and there are few options available to treat these infections (2, 6-10). Moreover, pioneering work has shown that several MBL inhibitors are able to potentiate imipenem against MBL-containing bacteria such as P. aeruginosa, indicating that the combination of a β-lactam with an MBL inhibitor would be clinically useful (11, 12). However, because of low amino acid sequence homology among MBLs, the spectrum of activity of current inhibitors varies considerably among enzymes.

In order to obtain broad-spectrum MBL inhibitors, one can simultaneously exploit two strategies (or employ them separately). The first strategy is to use coordination chemistry based drug design. Because all X-ray structures of MBLs indicate a tightly bound zinc ion(s) at the active site and site-directed mutagenesis studies show the zinc(s) are essential for the activity of the enzymes, in some embodiments universal MBL inhibitors contain a Zn(II) binding group that can interact with the central metal ion(s) strongly. The inventors have synthesized a library of compounds containing a number of Zn(II) chelating groups and found four lead inhibitors with Kᵢs of 3-19 μM. A second strategy involves the design of inhibitors that mimic the structures of the substrates, i.e., β-lactam antibiotics. The current challenge to find a universal MBL inhibitor is that these enzymes share a low homology (<25%) even within the same subclass. Because MBLs are able to hydrolyze most β-lactams (except rarely used monobactams), in some embodiments universal MBL inhibitors are substrate-like molecules. In fact, the most potent inhibitor obtained in studies by the inventors is a penicillin-like molecule. Moreover, a benefit of the β-lactam-like inhibitors is that they could be specific to MBLs, thereby having less toxicity to humans.

In some embodiments, there is design and development of a series of compounds based on a thiazolidine MBL inhibitor, which is a potent compound in initial studies and, importantly, meets both requirements of the compound having a strong Zn(II) chelating group and mimicking the structure of penicillin (for example). The activity of these compounds against a number of MBLs is tested and (quantitative) structure activity relationships (SAR) is analyzed and used to design compounds with improved activity. The ability of the novel MBL inhibitors to restore the susceptibility of β-lactam resistant bacteria is tested. In addition, x-ray crystallographic studies of metallo-β-lactamases, IMP-1 and Bla2, complexed with the novel inhibitors is performed. The exact binding modes of the novel lead inhibitors can provide valuable information on how to design compounds with improved activity. Moreover, activities on other metalloenzymes, e.g., matrix metalloproteinases (another class of Zn²⁺ dependent hydrolases), and human cell growth is examined to evaluate their selectivity as well as toxicity.

In some embodiments, another series of compounds is designed and developed based on a compound identified in preliminary studies. One can design and synthesize novel bicyclic compounds that not only closely mimic the structures of β-lactams, but have a known Zn²⁺-binding group. The enzymatic inhibition and cell activities of these compounds are tested. SAR and/or QSAR (quantitative structure activity relationship) studies as well as x-ray protein crystallography are used to design more active compounds.

### III. METALLO-β-LACTAMASES AND INHIBITORS THEREOF

The present invention concerns compositions and methods that employ inhibitors that target metallo-β-lactamases (MBL), including those in Gram positive or Gram negative bacteria. The inhibitors may be of any kind, although in specific embodiments of the invention one or more specific inhibitors are provided herein.

β-Lactam antibiotics are a broad class of antibiotics, each of which contains a β-lactam nucleus in their molecular structures. The class comprises at least penicillin derivatives (penams), cephalosporins (cephems), monobactams, and carbapenems, for example. In at least most cases, β-lactam antibiotics' mode of action includes inhibition of cell wall biosynthesis in the bacteria, for example. Thus, in some embodiments of the invention, the bacteria that are targeted are those that comprise at least one MBL. Metallo B-lactamases are able to hydrolyze at least penicillins, cephalosporins, and carbapenems, for example.

IMP-type carbapenemases in enteric Gram-negative organisms and in Pseudomonas and Acinetobacter species, for example, are encompassed in embodiments of targets of one or more inhibitors of the invention. VIM (Verona integron-encoded lactamases), such as those in P. aeruginosa, P. putida, P. fluorescens, P. mendocina, Pseudomonas stutzeri, and Enterobacteriaceae are included in embodiments of targets of one or more inhibitors of the invention. NDM-1 (New Delhi metallo-β-lactamase) are present in Escherichia coli and Klebsiella pneumonia, for example, and are included in embodiments of targets of one or more inhibitors of the invention.

In specific embodiments of the invention, Gram-negative bacteria such as Pseudomonas aeruginosa, Pseudomonas putida, Pseudomonas mendocina, Pseudomonas stutzeri, Klebsiella pneumoniae, Klebsiella oxytoca, Proteus mirabilis, Pseudomonas fluorescens, E. coli, Acinetobacter baumanii, Stenotrophomonas maltophilia, Bacteroides fragilis, Serratia marcescens, Klebsiella pneumoniae, Enterobacter aerogenes, Enterobacter cloacae, Shigella flexneri, Aeromonas hydrophila, Aeromonas caviae, Citrobacter freundii, Alcaligenes xylosoxidans, and/or Proteus vulgaris are targeted in the invention, because they are known to contain MBLs (also see Sanchez 2009, Assay and Drug Development Technologies; Maltezou 2009, International Journal of Antimicrobial Agents; Cornaglia 2001, Lancet Infectious Disease).

In specific aspects of the invention, one or more inhibitors are employed with imipenem, although in some embodiments they are used with older penicillins, such as amoxicillin, ampicillin or ticarcillin, for example. In some embodiments, one or more inhibitors are employed with cephalosporins, such as cephalexin, cephalothin, cefotaxime, or ceftazidime, for example.

The IMP-1 and several other metallo-beta-lactamase inhibitors may be present on plasmids and can move among bacteria by gene transfer. Therefore, the skilled artisan recognizes that routine methods in the art may be employed to combat a wide variety of bacterial pathogens that contain a metallo-enzyme. As such, the range of bacteria that are relevant to treatment with a beta-lactam and beta-lactamase inhibitor is expansive. In addition, metallo-beta-lactamases are also found in Gram positive bacteria, although this is not currently a major source of resistance. Gene transfer of metallo-beta-lactamases, however, could result in a treatment problem for Gram positive bacterial infections.

### IV. PHARMACEUTICAL PREPARATIONS

In some embodiments of the invention, one or more inhibitors of a metallo-β-lactamase (MBL) is delivered to an individual. In particular embodiments, the MBL(s) are provided to the individual at or near the same time as an antibiotic that targets a bacterial that has at least one MBL. The inhibitor and the antibiotic may be delivered simultaneously, although in some cases the inhibitor is provided to the individual prior to and/or subsequent to delivery of the antibiotic. The inhibitor and antibiotic may be delivered to the individual using the same or different delivery methods.

The skilled artisan recognizes that the dosage of antibiotic plus the inhibitor depends on patient age and weight, type of bacterial infection, other medications, etc, but there are routine methods in the art to determine the dosage. The ratio of antibiotic to inhibitor may be determined. In specific embodiments, the ratio of antibiotic to inhibitor in specific embodiments is or is at least 0.5:1, 0.75:1, 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, and so forth.

Pharmaceutical compositions of the present invention comprise an effective amount of one or more compositions of the invention dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition of the invention or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The MBL inhibitor may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The MBL inhibitor may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include MBL inhibitor, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the PKR inhibitor may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the composition(s) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. KITS OF THE INVENTION

Any of the compositions described herein may be comprised in a kit. In a non-limiting example, a metallo-β-lactamase (MBL) inhibitor is comprised in a kit in a suitable container means. A β-lactam antibiotic may or may not be included in the same kit, however. In some embodiments, the kit comprises the inhibitor and the antibiotic formulated in the same formulation or separate formulations. The inhibitor and/or antibiotic formulations may be of any suitable kind, including pill, tablet, cream, aerosol, liquid, and so forth. In some embodiments, the kit comprises an apparatus for extracting a sample from an individual suspected of having a bacterial infection (though in some cases the individual is known to have an infection); such an apparatus may comprise, for example, a swab, scalpel, syringe, and so forth.

The components of the kits may be packaged either in aqueous media or in lyophilized form, for example. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the MBL inhibitor and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The composition may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. In some embodiments, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Coordination Chemistry Based Approach Leads to Novel Potent IMP-1 Inhibitors

Following rational design and synthesis of MBL inhibitors, as well as x-ray protein crystallography, there are several exemplary inhibitors of IMP-1, with Kᵢs ranging from 3-19 μM, using a similar coordination chemistry based drug design.

The existing MBL inhibitors have largely been identified from high-throughput screens and their potencies vary considerably for the different MBLs, even within the same subclass, possibly because of low sequence homologies among the enzymes. There have been rational design efforts, however, focused on using thiol as a Zn-binding group (38), which, although very effective (as it has a great affinity for Zn²⁺), suffers from high chemical and metabolic instability. In order to obtain broad spectrum MBL inhibitors with favorable pharmacokinetic properties, the inventors reasoned that at least in certain embodiments the drug design is focused on the central metal ion(s), using a coordination chemistry-based approach.

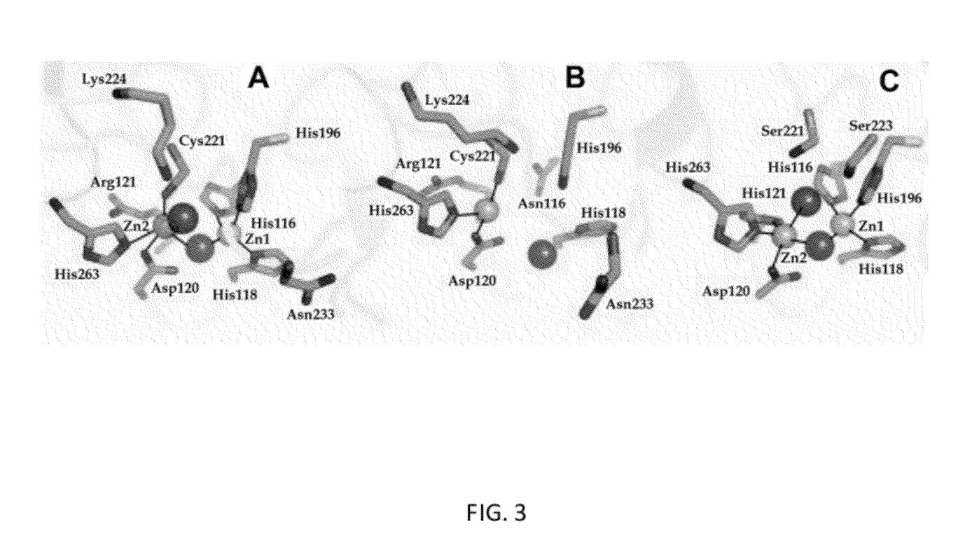

In certain aspects to the invention, because Zn ion(s) at the active site are common to all MBLs and play an essential role for catalysis (FIG. 3), compounds containing a strong Zn(II) chelating group are useful inhibitors. The coordination chemistry-based lead inhibitors are developed further to have improved activity as well as selectivity/specificity for metallo-β-lactamases, for example by using a structure and/or substrate based drug design approach. The inventors thus synthesized a library of 36 compounds comprising 20 Zn(II) chelating/binding groups, together with a simple hydrophobic side chain (e.g., phenyl or benzyl). Twenty compounds, each having a different metal binding group, are shown below:

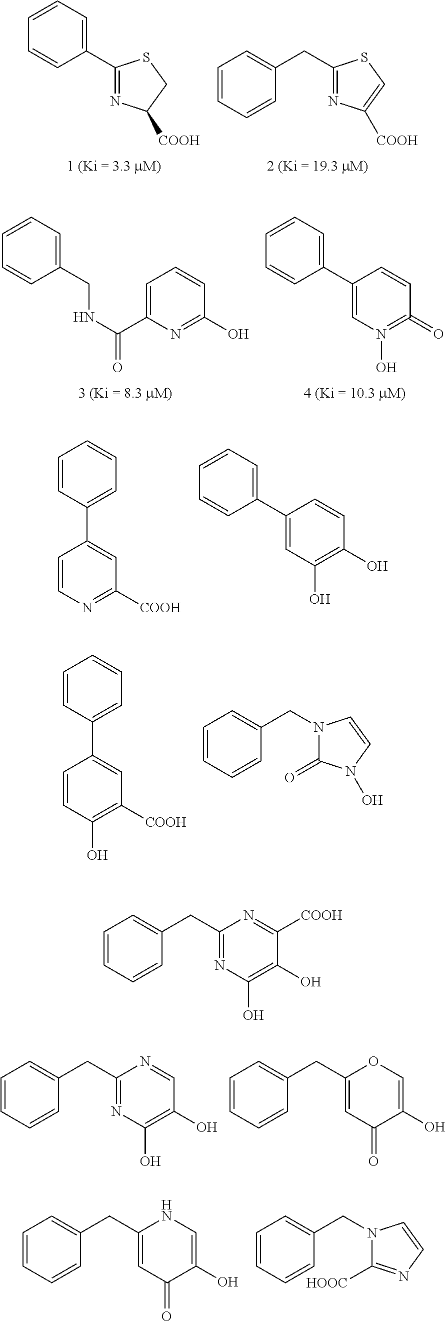

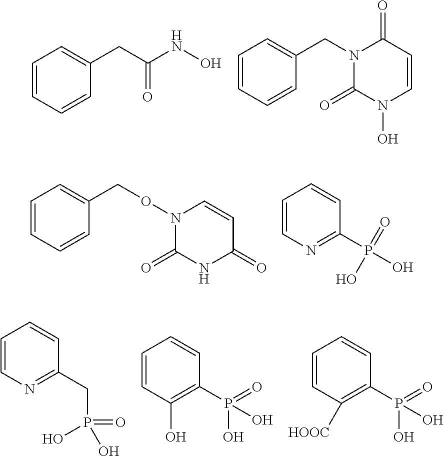

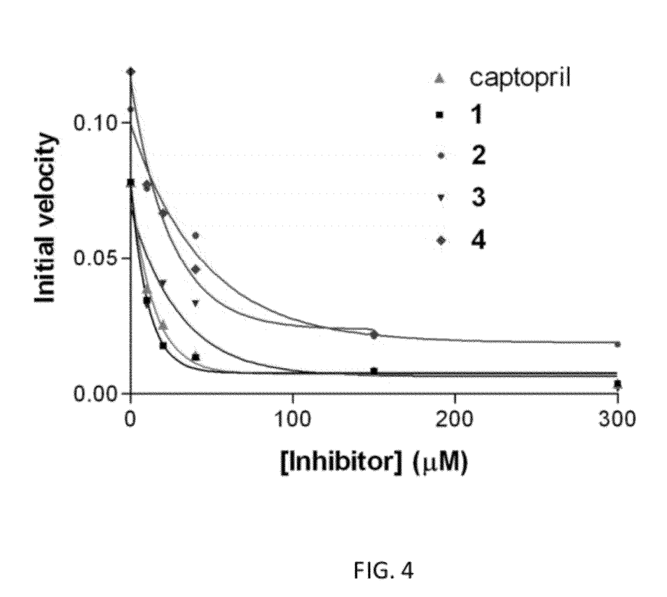

In certain embodiments, this focused compound library is to identify Zn(II) chelating/binding groups that work well for metallo-β-lactamases. The activity of these compounds was tested against a representative MBL, IMP-1, which is also of clinical importance (2, 55). The assay was carried out by using 2 nM enzyme and 20 μM nitrocefin as a substrate (Kₘ=25 μM) in 50 mM HEPES. Compounds 1-4 were found to be good lead inhibitors with Kᵢs ranging from 3-19 μM, with activities comparable to a known inhibitor captopril (Kᵢ=4.7 μM), which is the positive control in the assay. The dose responsive curves used to determine IC₅₀s/Kis are shown in FIG. 4.

The coordination chemistry based approach was quite effective: four lead inhibitors were identified among a relatively small, rationally designed compound library. In addition, these drug-like compounds re readily synthesized and are therefore amenable to further modification. In certain embodiments, they serve as scaffolds for further design and development (see Examples below).

### Example 2

### X-Ray Crystallographic Studies

The further development of lead compounds is facilitated by the availability of X-ray crystal structures of the target enzyme in complex with the compound. The wild type IMP-1 structure was reported previously (56). A Cys221Gly mutant of IMP-1 was expressed, purified and crystals were grown by the hanging drop method at 25° C. in 0.8M di-sodium hydrogen phosphate, 0.8M di-potassium hydrogen phosphate, 0.1M HEPES, pH 7.5. Crystals were observed within one week and X-ray data was obtained to a resolution of 1.7 Å. A structure is available. In addition, the wild type IMP-1 enzyme has also been crystallized in 100 mM sodium citrate pH 5.5 with 40% PEG 600 and the crystals are characterized by standard means (the crystals diffract to 2.0 Å). One can soak the IMP-1 crystals with the lead compounds to obtain their IMP-1 complex structures. Crystallization screens can be performed in the presence of the compound to obtain a co-crystal structure.

The Bacillus anthracis Bla2 metallo-β-lactamase has been expressed from E. coli, purified, and kinetic parameters for β-lactam hydrolysis have been performed (59). In addition, the Bla2 crystal screens were performed and the enzyme crystallized from 25% PEG 6000 containing 0.05 M imidazole using the hanging drop method and a structure has been determined to 2.3 Å. It has also been found that these native Bla2 crystals lend themselves to soaking with previously identified inhibitors captopril and glutathione, and diffract to 2.3 Å in the presence of either compound. One can solve the structure of these complexes by standard means. The Bla2 enzyme is also used for soaking and/or co-crystallization experiments with the exemplary lead compounds described herein.

### Example 3

### Exemplary Research Design and Methods

One can rationally design and develop substrate (β-lactam)-like compounds that contain a strong Zn(II) chelating group and one can carry out x-ray crystallographic studies of these inhibitors, in order to obtain compounds with improved activity as well as specificity. A coordination chemistry based design approach has yielded good lead inhibitors (e.g., compounds 1-4). Because there are no broadly active metallo-β-lactamase inhibitors available, further development of these compounds using novel drug design strategies are needed. Given the rapid worldwide spread of metallo-β-lactamase genes in many bacterial species and the adverse consequences this may cause, clinically useful MBL inhibitors that are able to restore the antibacterial activity of β-lactams are needed.

Development of Thiazolidines and Analogous Compounds as MBL Inhibitors

In this embodiment, there is use of medicinal chemistry, quantitative structure activity relationships (QSAR) and x-ray crystallography to design, synthesize and test novel thiazolidine compounds targeting a broad spectrum of clinically important metallo-β-lactamases. The major drawbacks of current MBL inhibitors are chemical/metabolic instability (e.g., thiol based inhibitors) and narrow spectrum of activity (e.g., inhibitors resulting from high-throughput screening), which make them less feasible to be used clinically. In some embodiments, the strategies to overcome these problems are to target two common structural features of all MBLs (or at least the clinically important subclass B1 enzymes): 1) Zn ion(s) at the active site and 2) wide substrate promiscuity. Inhibitors that mimic the structures of β-lactams could have good selectivity to MBLs, rather than other Zn(II) dependent enzymes, as human enzymes do not recognize β-lactams. Therefore, these compounds exhibit less toxicity and/or side effects, in specific embodiments.

One can design and synthesize a series of thiazolidines and their analogs based on the core structure of compound 1, a potent lead inhibitor discovered in preliminary studies. In addition, because of the structural similarity between 1 and penicillin, structures of β-lactams are used to guide further drug design, which result in potent inhibitors with broad activities against MBLs, in at least certain cases. The biological activity of these compounds is then tested and SAR and QSAR is performed in order to design compounds with improved activity. In another embodiment, one can carry out x-ray crystallographic studies of IMP-1 in complex with these novel inhibitors. Knowledge of how the inhibitors interact with the Zn2+ ion(s) as well as other regions of the active site greatly facilitates rational drug design.

Drug Design and Synthesis Based on Compound 1.

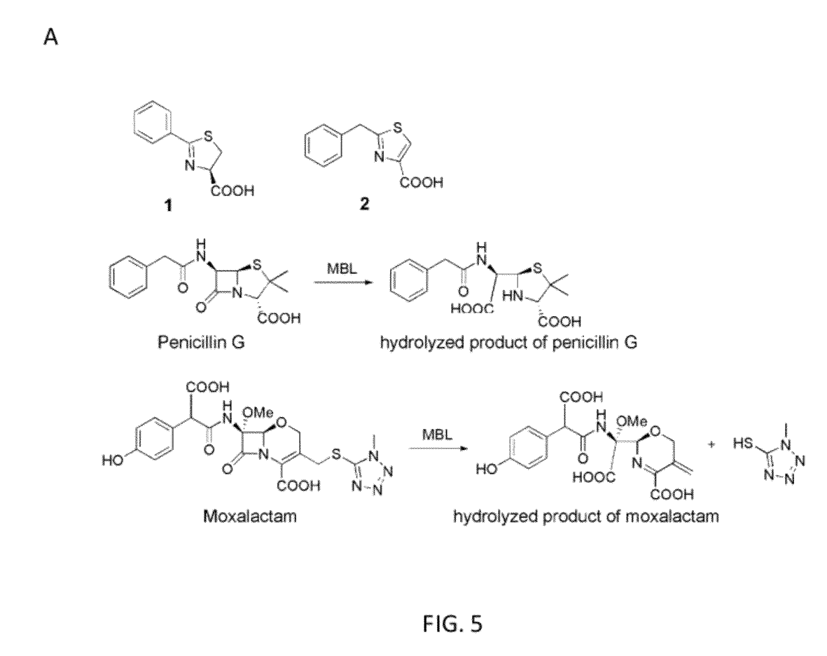

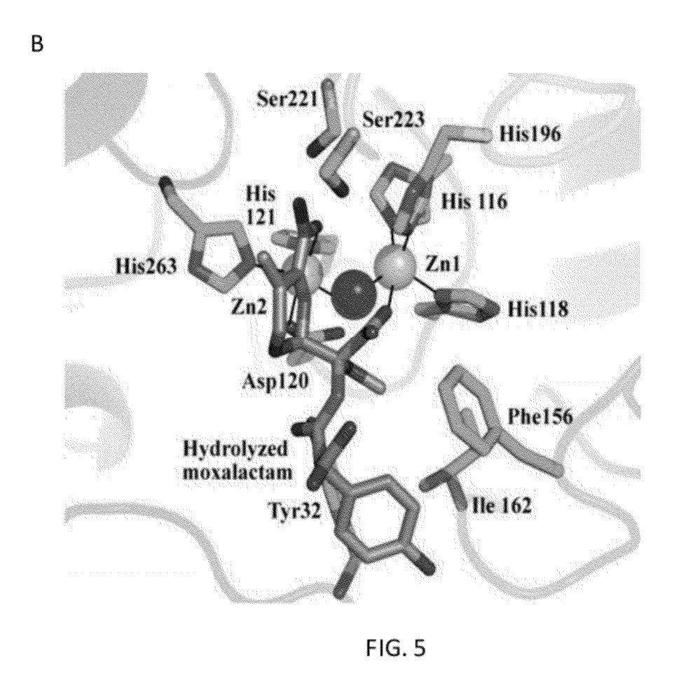

The design of compound 1 and its analog 2, at least in part, originated from their structural similarity to penicillins (or more accurately to the hydrolyzed product), as shown in FIG. 5A. In addition, in certain embodiments the potent activity of 1 is not serendipitous, but may stem from this similarity. One piece of supporting evidence is the crystal structure of Stenotrophomonas maltophilia L1 MBL complexed with the hydrolyzed product of moxalactam (FIG. 5A), where an imino (C═N) and a carboxylate group from the antibiotic chelate the Zn2 ion while the second carboxylate resulting from hydrolysis interacts with Zn1 (60), as shown in FIG. 5B. Although the S. maltophilia L1 enzyme is a class B3 MBL, its active site contains a similar di-Zn(II) metal center. Moreover, the complex of a class B2 metallo-β-lactamase CphA from Aeromonas hydrophila with a hydrolyzed carbapenem antibiotic, biapenem, also shows that the N and the carboxylate group serve to chelate the Zn2 (61). Thus, in some embodiments the imino and the carboxylate groups of compound 1 also act as Zn2 chelators in IMP-1. However, a crystallographic study can be carried out to obtain the x-ray structure of IMP-1 in complex with 1 and 2, as described below.

Modifications based on compound 1 can be made to study its structure activity relationships (SAR). In addition, analogous compounds that better mimic the structures of β-lactams can be synthesized in an effort to optimize the activity. Moreover, it is noteworthy that, unlike β-lactams, synthesis of these compounds is straightforward and provide good yields (described below), with the main step involving the condensation of cysteine with a nitrile or an aldehyde, all of which are readily available commercially. The ease of synthesis makes further development of this class of compounds attractive.

In at least some cases, the first step is to investigate how the degree of aromaticity of the thiazolidine ring affects the potency of inhibition. Compound 1 is ˜6× more active than 2. The enhanced activity of 1 could be due to a higher coordination capability of the imino group in 1 to Zn²⁺, compared to the nitrogen atom of the aromatic thiazole ring in 2, as the delocalization effect of aromaticity will decrease the electron density of the N lone pair. Another possible reason is the difference of the phenyl side chain in 1 versus the benzyl in 2. Consequently, the following compounds will be made to facilitate a side-by-side comparison:

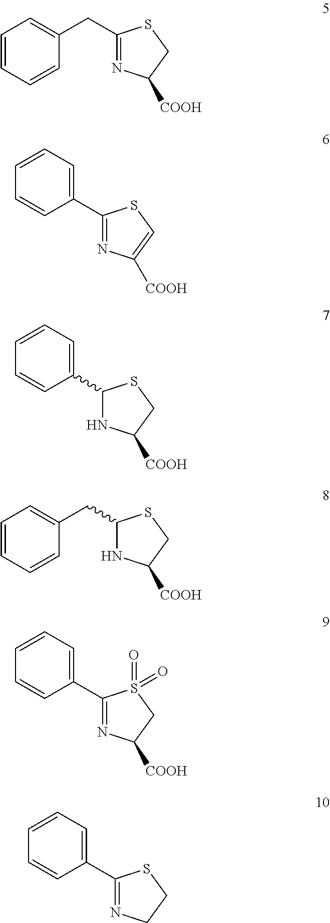

Compounds 7 and 8 without any double bond in the ring are included in the set, although in these cases the N atom (a more electronegative atom) will no longer obtain electrons from the S atom through conjugation. In addition, oxidation of compound 1 can lead to sulfone analogs 9. β-Lactam sulfones (e.g., sulbactam and tazobactam) are used as potent serine β-lactamase inhibitors. Here, the sulfone, a strong electron-withdrawing group, will result in fewer electrons on the N atom and this effect may be tested. Compound 10 lacking a 3-carboxylate group can also be synthesized to examine the importance of this group.

In the second step, since the stereochemistry of C3 of the thiazolidine ring (according to the nomenclature of penicillins) in 1 is inversed from that of penicillin (FIG. 5A), causing a different orientation of the important carboxylate group, the enantiomers 11-14 (shown below) of compounds 1, 5, 7 and 8 can be synthesized, starting from commercially available D-cysteine and their activities can be tested. Since their carboxylate groups will have the same orientation as that of penicillin, improved potency is observed in at least certain cases.

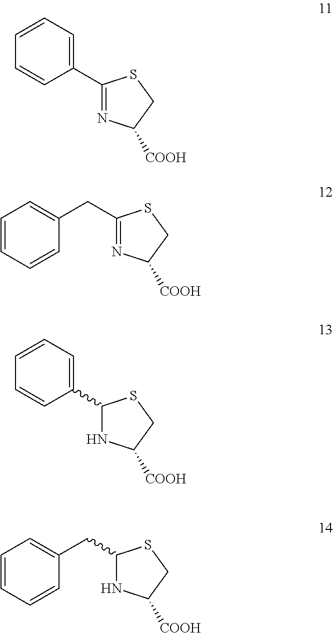

Thirdly, the effects of replacing the thiazolidine ring with another analogous ring will be examined and the following compounds will be synthesized and tested:

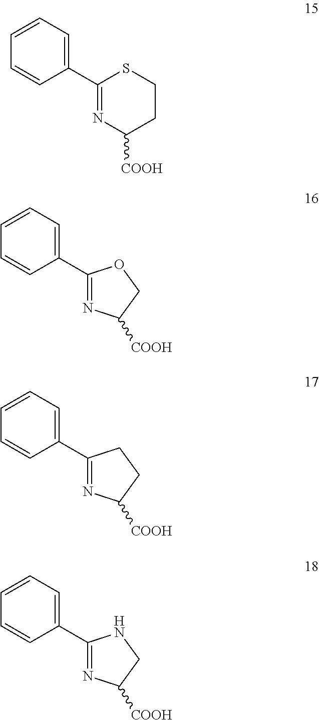

The rationale is that because, in addition to penicillins that have a similar tetrahydrothiazole ring, cephalosporins and carbapenems contain other rings, e.g., a dihydrothiazine ring and can also be investigated. Moreover, cephalosporins (e.g., nitrocefin) and carbapenems (e.g., imipenem) generally bind to MBLs more tightly than penicillins with reduced Kₘ values, as shown in Table 1.

TABLE 1
Kₘ values of selected antibiotics for MBLs. Data are from ref. 2.

| | IMP-1 | VIM-1 | GIM-1 | SPM-1 |
|---|---|---|---|---|
| | Km (μM) | Km (μM) | Km (μM) | Km (μM) |
| Penicillin | 520 | 841 | 46 | 38 |
| Ampicillin | 200 | 917 | 20 | 72 |
| Nitrocefin | 27 | 17 | 5.8 | 4 |
| Cephalothin | 21 | 53 | 16 | 4 |
| Imipenem | 39 | 1.5 | 27 | 37 |
| Meropenem | 10 | 48 | 2.7 | 281 |

For example, penicillin G (Kₘ: 520 μM) binds to IMP-1 ˜20× less tightly than nitrocefin (Kₘ: 27 μM) and ˜13× less tightly than imipenem (Kₘ: 39 μM). Although the difference in binding affinity may also originate from the second sidechain of cephalosporins and carbapenems (which is discussed below), it is useful to carry out a SAR study with different ring systems.

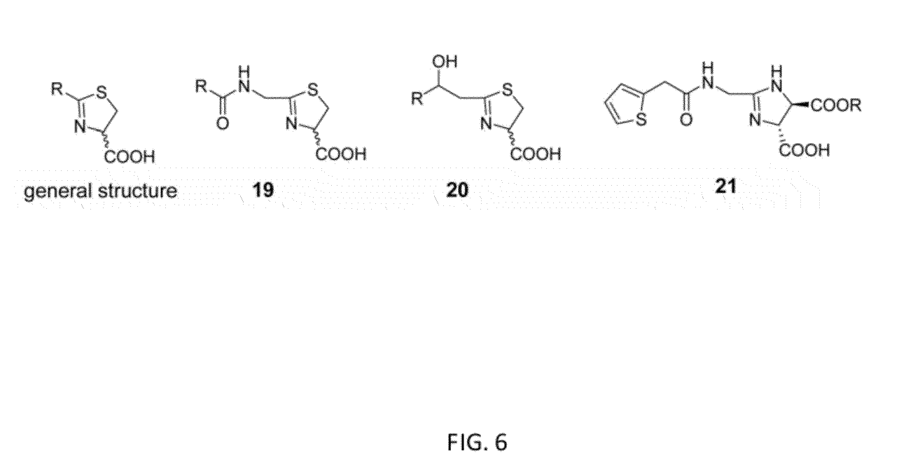

Finally, an expanded library (˜50 compounds) is synthesized to study the structure activity relationship with respect to the sidechain of 1. The general structure of this compound library and several representative compounds to be made are shown in FIG. 6. The affinity of compound 1 (Kᵢ: 3.3 μM) to IMP-1 is ˜160×, 8×, and 10× better than penicillin G, nitrocefin and imipenem, respectively. Therefore, in some embodiments one can further develop this class of compounds, given the ease of their synthesis. In addition, the sidechain of 1 can be optimized. As discussed above, structures of β-lactam antibiotics can be used to guide design. For example, the common side chains of penicillins and cephalosporins include a variety of 6-arylacetamido groups. Compound 19 can therefore be synthesized with a series of R groups. Similarly, compound 20, whose sidechain derives from imipenem, can be synthesized with a variety of R groups. Moreover, since the Kₘ values of cephalosporins and carbapenems are lower than penicillins (Table 1), which may indicate a benefit of having another sidechain at the 2-position of the thiazolidine ring, compounds based on 21 can be synthesized to examine this.

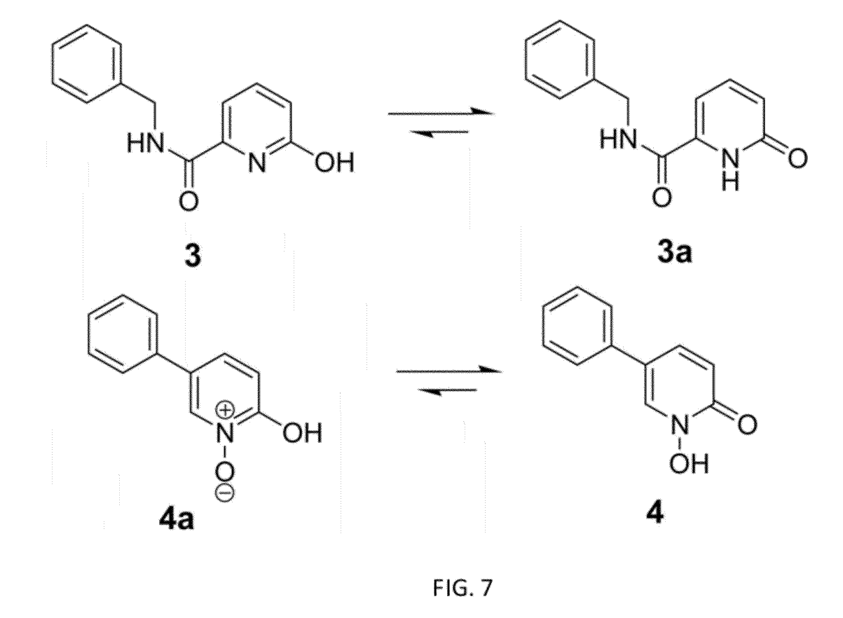

In addition, quantitative structure activity relationship (QSAR) studies are used to optimize the activity of the sidechain of 1 to complement the above design approach. A collection of 30-50 commercially available nitriles will be purchased and used to generate a random thiazolidine compound library, whose general structure is shown in FIG. 7. The side chains of these nitriles are selected from various electrostatic, steric, hydrophobic as well as H-bond donor/acceptor features. The activities of these compounds are tested against IMP-1 and the data are used to carry out a 3D-QSAR study, using the comparative molecular similarity index analysis (CoMSIA) (62) and/or comparative molecular field analysis (CoMFA) (63) methods. Using these QSAR results, in which activity is related to 3D-structure in a quantitative manner, one can optimize the electrostatic, steric, hydrophobic and H-donor/acceptor field requirements for MBL inhibition.

Synthesis of thiazolidine (e.g., 1) and tetrahydrothiazole (e.g., 13) compounds can be readily achieved by a single step condensation of cysteine (L-, D- or DL-) and a nitrile (for thiazolidine) and an aldehyde (for tetrahydrothiazole) in generally good yields, as shown representatively below:

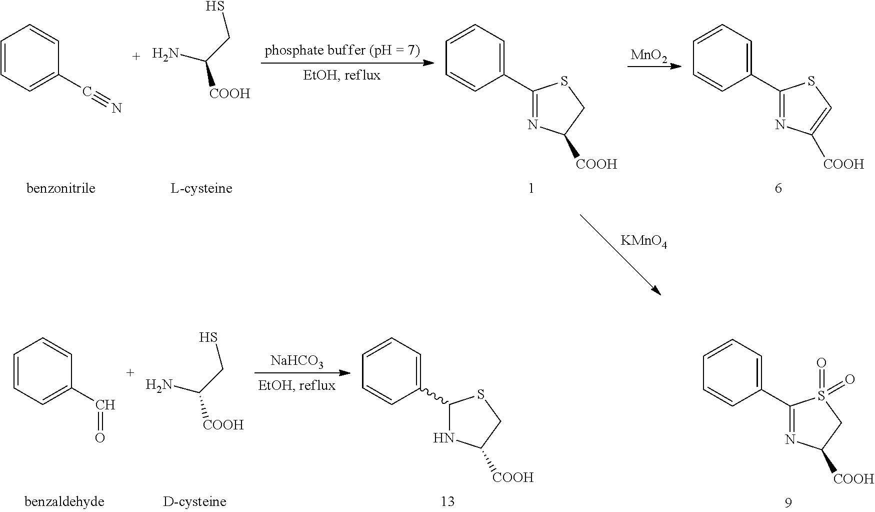

Oxidation of thiazolidine (e.g., 1) by activated MnO₂ can give aromatic thiazole compounds (e.g., 6), and by KMnO₄ can afford sulfone compounds such as 9. For the synthesis of compounds with a different ring, cysteine can be replaced by an analogous starting material, such as homocysteine (for 15), serine (for 16) and 3-amino-alanine (for 18).

Biological Activity Testing

Enzymatic assays. The subclass B1 metallo-β-lactamase IMP-1 from Pseudomonas aeruginosa can be used as the primary screen. This enzyme is encoded on a transferable plasmid that has been widely disseminated. IMP-1 is therefore of clinical importance (55). An E. coli expression system has previously been developed for the IMP-1 enzyme (58) and has recently been modified to including an affinity tag. The IMP-1 enzyme is expressed from an IPTG-inducible promoter in E. coli and a periplasmic extract is obtained by osmotic shock. The enzyme has an 8-amino acid strep-tag that allows for purification on a commercially available affinity resin. A second fractionation is accomplished by gel filtration chromatography with a high-resolution G-75 column. Analysis of the protein stability and kinetic parameters for the IMP-1 strep-tag enzyme indicate there is no change in function or stability relative to the un-tagged enzyme.

The inventors have established a screening assay method for this enzyme using nitrocefin as a substrate, whose hydrolyzed product by a MBL has a strong optical absorption at 482 nm. The hydrolysis process can therefore be conveniently monitored at this wavelength using a microplate reader/photometric spectrometer. The assay can be carried out in a 96-well microplate using 2 nM enzyme, 20 μM nitrocefin (Km=25 μM), 50 mM HEPES buffer (pH=7.5), and 20 μg/mL BSA. The initial velocities of wells containing increasing concentrations of an inhibitor will be calculated. The IC₅₀ values as well as Kᵢs can be calculated using standard non-linear regression fitting (64).

Potent inhibitors against the P. aeruginosa IMP-1 enzyme can be further evaluated against a panel of 4 metallo-β-lactamases, including Bacillus anthracis Bla2 (B1), P. aeruginosa VIM-2 (B1), A. hydrophila CphA (B2) and S. maltophilia L1 (B3). These enzymes cover all three subclasses of MBLs and include two transferable enzymes, IMP-1 and VIM-2, which are most prominent in a clinical setting. The inventors developed the Bla2 expression system and have obtained the VIM-2 gene for protein expression (59). The CphA and L1 systems can be obtained for expression of these enzymes as well. The assay conditions for each of these enzymes can be optimized and used to test the activities of our compounds. In at least some embodiments wherein the inhibitors exhibit activities against this broad range of metallo-β-lactamases, they are clinically useful.

Bacterial growth inhibition assays. Next, the compounds that display inhibition in the enzyme assays can be tested for the ability to restore antibacterial activity of β-lactams on MBL producing bacteria. This is a critical assessment for the potential effectiveness of the inhibitors. Thus, the minimal inhibition concentrations (MIC) of ampicillin and imipenem towards an E. coli laboratory strain containing the IMP-1 gene on a plasmid can be first tested in the absence of MBL inhibitors (57, 58). The minimal inhibition concentration is defined to be the smallest concentration of imipenem that can inhibit the visible bacterial growth. The MICs of ampicillin and imipenem in the presence of a variety of concentrations of the inhibitors can then be tested. In specific embodiments, a useful MBL inhibitor significantly decreases the ampicillin and imipenem MIC for the bacterial strain. In addition, the inhibitors can be tested on an imipenem resistant Pseudomonas aeruginosa strain that is known to contain the IMP-1 gene. Ideally, an inhibitor compound can significantly decrease the P. aeruginosa MICs for ampicillin and imipenem. In some embodiments, some compounds reduce the MIC of E. coli containing IMP-1 but not the P. aeruginosa strain. Such a result could be due to multiple factors but in at least some embodiments it would be related to decreased permeability of P. aeruginosa. Going forward, the focus would be on those compounds that exhibit potent activity on E. coli and P. aeruginosa.

Toxicity screening using human enzymes and cells. A potential pitfall of inhibitors of Zn(II) dependent enzymes is their selectivity/specificity. A non-selective inhibitor could have serious toxicity and/or side effects on humans, since metalloenzymes play pivotal roles in many important cellular processes.

Two strategies can be used to manage this potential problem. First, as described above, because human enzymes generally do not recognize β-lactams and they have been used for many years as safe drugs, one can design MBL inhibitors that mimic the structures of β-lactams, such as the thiazolidine inhibitors (e.g., 1 and 2) described herein. These inhibitors could therefore be specific to MBLs. Indeed, using a commercially available assay kit obtained from Enzo (for example), compounds 1 and 2 did not exhibit inhibition of human matrix metalloproteinase 8 (MMP-8) at concentrations up to 100 μM. Second, the most potent MBL inhibitors identified herein can be tested against three Zn(II) dependent enzymes, including human MMP-8, human angiotensin converting enzyme 1 (ACE-1) and human histone deacetylase 1 (HDAC-1). Each of these represents a distinct class of Zn(II) enzymes: MMPs are metallo-endoproteases; ACE-1 is a Zn dependent carboxypeptidase; and HDACs hydrolyze the acetyl group from the acetylated lysine sidechain of a large collection of proteins. In addition, the activity of these enzymes can be readily assessed, as there are commercially available assay kits (e.g., from Enzo).

Moreover, the activity of the lead inhibitors can be tested for their impact on the growth of two human non-cancerous cell lines, Beas2B (lung epithelial) and WI-38 (fibroblast), in order to evaluate their potential toxicity (54). 1×10⁵ cells can be inoculated into each well of a 96-well plate and cultured Dulbecco's Modified Eagle's Medium (DMEM) supplemented with 10% fetal bovine serum at 37° C. in a 5% CO₂ atmosphere with 100% humidity overnight for cell attachment. After addition of compounds to each well, plates can be incubated for 1 day after which cell viability can be assessed by the [3-(4,5-dimethylthiazol-2-yl)-5-(3-carboxymethoxyphenyl)-2-(4-sulfophenyl)-2H-tetrazolium, inner salt (MTS) assay (for example), using a commercially available kit (Promega). IC₅₀s of each compound can be calculated from dose response curves.

X-Ray Crystallographic Studies of IMP-1

Because the crystal structures of IMP-1 complexed with several small molecule compounds have been solved and published to provide valuable insights into the binding mode of the compounds (12, 41, 65, 66), one embodiment to obtain information on the structure of the compounds with IMP-1 would be to use computational docking programs. However, in some embodiments there are limitations with this approach that may impede its use with MBLs. First, the conformation of the MBL active site is known to be highly flexible. In particular, the so-called flap consisting of the residues 22-31 of IMP-1 is an important part of the active site and acts as a “cap” that is open when there is no ligand (substrate or inhibitor) and closed when a ligand is bound to the enzyme (56). There is a very large rms difference (˜3.5 Å) when an inhibitor is bound to the enzyme. Moreover, a different ligand can also cause a different conformation of the flexible flap (41). This high flexibility is important for enzyme function, as it allows MBLs to recognize and hydrolyze essentially all β-lactams. However, this feature makes structure based drug design a great challenge. The inhibitors found in the preliminary studies have very different structures from the inhibitors whose crystal structures in complex with IMP-1 have been solved (12, 41, 65, 66). Because the current docking programs cannot deal with such a large conformational change, crystal structures of IMP-1 in complex with the inhibitors are therefore needed for future drug design.

In addition, although there are many crystal structures of metallo-β-lactamases complexed with a variety of inhibitors, only three structures show the binding modes as well as substrate recognition of β-lactam-like molecules: 1) S. maltophilia L1 MBL complexed with the hydrolyzed product of moxalactam (60) (shown in FIG. 5A) 2) A. hydrophila CphA with the hydrolyzed biapenem (61) and 3) the B1 subclass NDM-1 β-lactamase complexed with hydrolyzed ampicillin (Zhang, H. and Hao, Q. (2011). Crystal structure of NDM-1 reveals a common β-lactam hydrolysis mechanism. FASEB J. 25: 2574-2582.). In addition, the potent inhibitors 1-3, especially 1, are actually substrate-like (product-like) inhibitors and their crystal structures bound to IMP-1 are useful.

As described above, the inventors have obtained a crystal structure of an IMP-1 Cys221Gly mutant and crystals of wild type IMP-1. One can determine structures of wild type IMP-1 complexed with the inhibitors 1-3. The first set of studies include soaking IMP-1 crystals with the relevant compounds and then obtaining X-ray data to look for the presence of the compounds in the IMP-1 active site. If this method does not yield useful data, a solution of the IMP-1 protein and the relevant compounds are screened for crystallization conditions that yield a co-crystal structure.

The structure of the B. anthracis Bla2 enzyme can also be determined in complex with compounds that are found to inhibit this enzyme in addition to inhibition of IMP-1. The structure of wild type Bla2 has been solved and crystals with captopril and glutathione have been obtained that diffract to 2.3 Å. Therefore, the first studies can soak the crystals containing wild type Bla2 with the relevant compounds. If this approach is not successful, screening can be performed to identify conditions in which solutions containing Bla2 and the relevant inhibitor compounds co-crystallize.

MBL Inhibitors Based on the Structures of Compound 3 and β-Lactams.

One can use medicinal chemistry, QSAR and x-ray crystallography to design, synthesize and test novel thiazolidine compounds targeting a broad spectrum of clinically important metallo-β-lactamases. A second series of compounds can be developed based on the scaffold of compound 3, which is not only the second most potent inhibitor, but based on its structure, can lead to compounds that better mimic cephalosporins (one major class of β-lactam antibiotics) and therefore have improved activity, in at least some embodiments. In addition, novel monocyclic and bicyclic compounds can be designed and synthesized that not only mimic the structures of β-lactams, but also contain a known Zn²⁺ binding group that can interact with the central metal ion(s) strongly.

Drug Design and Synthesis Based on Compounds 3

The second most active lead compound discovered in preliminary studies, compound 3, can be considered a derivative of pyridine-2-carboxylic acid, which is also an inhibitor of the subclass B2 metallo-β-lactamase CphA from A. hydrophila with a Kᵢ value of 5.7 μM (67).

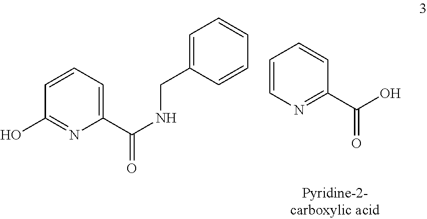

However, it exhibits little or no activity against other MBLs. The crystal structure of CphA complexed with its analog, pyridine-2,4-dicarboxylic acid, indicates that the N and the 2-carboxylate chelate the Zn2 ion at the active site (67), which is consistent with other crystallographic studies including that shown in FIG. 5B. Based on this structural information, in some embodiments 3 binds to MBLs (e.g., IMP-1) in a similar manner. However, there are considerable differences between subclass B2 enzymes (e.g., CphA) and clinically relevant subclass B1 enzymes (e.g., IMP-1) in both sequence and, maybe more importantly with respect to inhibitor design, the number of Zn ions at the active site. CphA has only one Zn ion at the Zn2 binding site, while B1 enzymes contain two Zn ions. Indeed, pyridine-2-carboxylic acid has only a very weak inhibitory effect on IMP-1. In addition, compound 3, unlike compound 1, has an additional 6-hydroxyl group adjacent to the N atom that can be tautomerized to 3a with an amide-like structure, as shown in FIG. 7. In addition, another good IMP-1 inhibitor (compound 4 from preliminary studies) also has a similar tautomerization mode (FIG. 7). The binding modes of 3 and 4 are thus less clear. An x-ray protein crystallographic study can be performed to determine how these two compounds bind to IMP-1. The structural information revealed from the study can facilitate rational design and development of these compounds, as is standard in the art.

In addition to the x-ray crystallographic study, small scale medicinal chemistry modifications of these two lead compounds can be performed in an effort to identify the key features that are responsible for the potent activity. The following analogs of compound 3 can be synthesized:

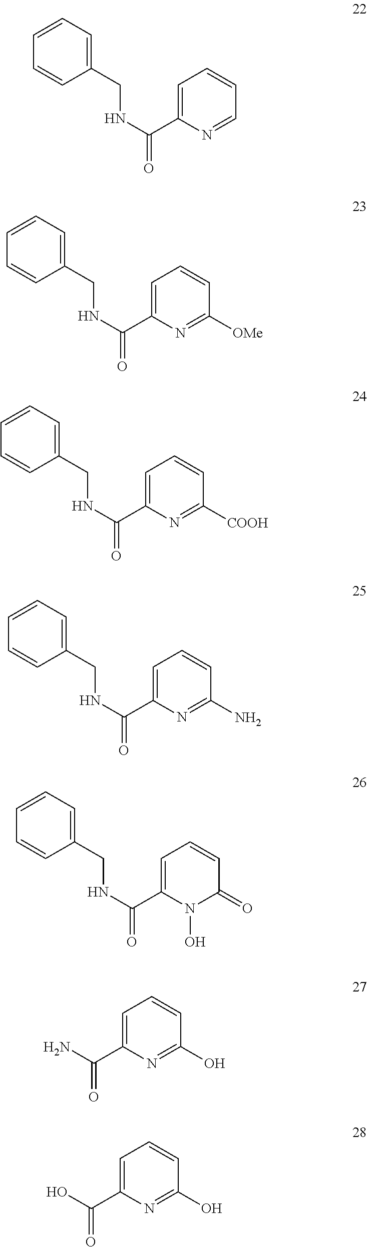

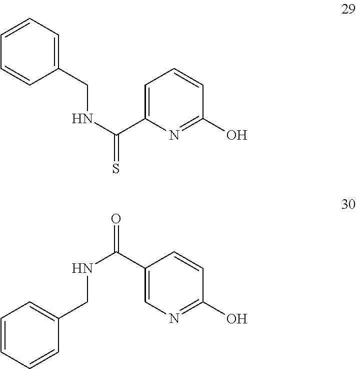

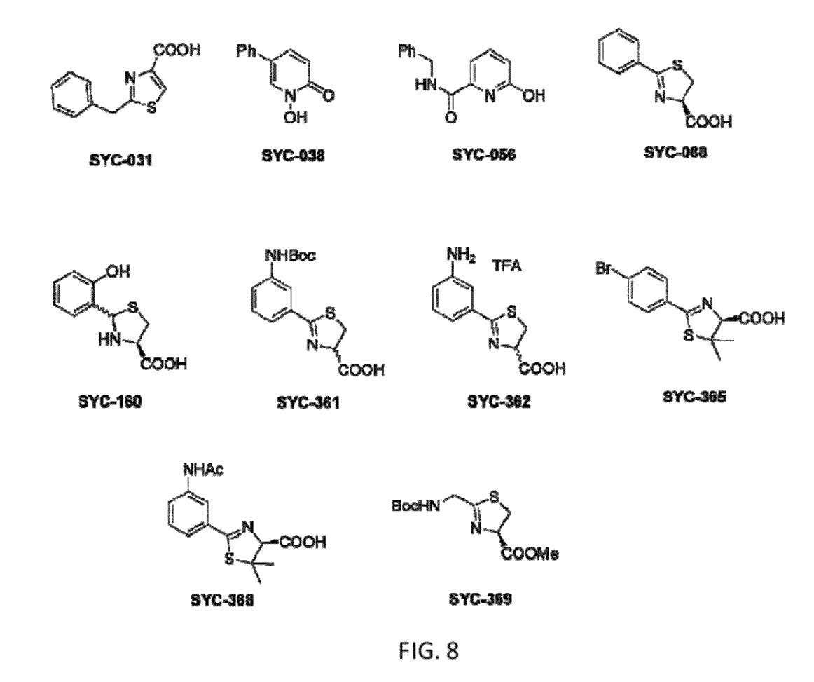

Compound 22 will test whether the 6-hydroxyl group is important. Compound 23 with a 6-methoxy is not able be tautomerized to a cyclic amide form as found in 3a and therefore will test the role of tautomerization (FIG. 8). In compounds 24 and 25, the 6-hydroxyl group is replaced by a carboxylate and an amino group, respectively. In some embodiments compound 24 is a very potent MBL inhibitor, as pyridine-2,6-dicarboxylic acid, a potent metal chelator, is known to potently inhibit the activity of IMP-1 and CphA (but not other MBLs) (67). However, a concern about this compound was that it may act as a Zn²⁺ scavenger, depleting the Zn ion from the active site, rather than tightly binding to the enzyme. In addition, its small size and potent chelating ability may result in non-selective metalloenzyme inhibition. Compound 24 with a large hydrophobic side chain, which mimics the structure of cephalosporins with a six-membered ring, is a better design, in some embodiments. Compound 26 is an oxidized form of 3, which resembles the structure of 4. The next series of compounds 27-30 focus on the amide side. Compounds 27 and 28 can test the importance of the hydrophobic benzyl group for the inhibition activity. Compound 29 has a thioamide group. The rationale for the design is that Zn²⁺, a softer metal ion, has a high affinity for soft ligands, such as thiols. Many known MBL inhibitors are in fact thiols. However, thiols can be easily oxidized. Thioketones and thioamides are much more stable and can also be good ligands for soft metal ions such as Zn²⁺. The activity of compound 30 with a 3-carboxylamide can reveal whether its counterpart in compound 3 participates in metal chelation.

These compounds can be easily synthesized with the major step being the well established method of amide (peptide bond) formation. Further design, synthesis and development based on these two lead compounds can depend upon the above structure activity relationship study as well as x-ray crystallography.

Novel β-Lactam Analogs as MBL Inhibitors

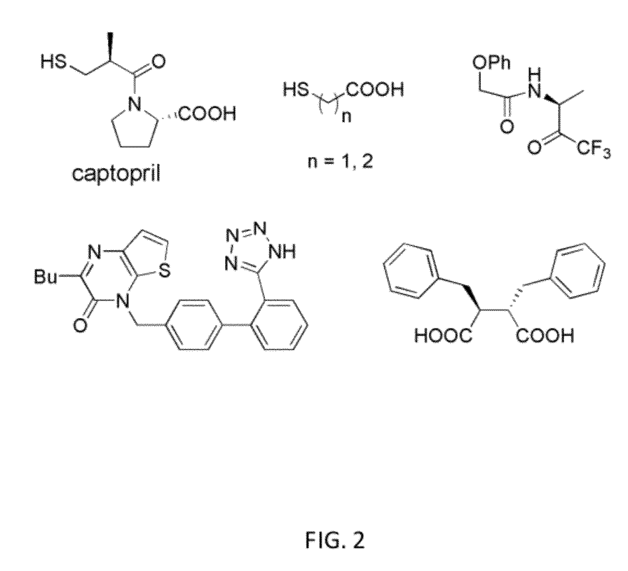

Phosphonate, phosphinate and activated ketone have proven to be effective Zn²⁺ binding groups in inhibiting Zn(II) dependent enzymes (e.g., matrix metalloproteinases). In addition, compounds containing a trifluoromethyl substituted ketone, which is one type of activated ketone, were found to be, in general, weak MBL inhibitors, with one example shown below (also in FIG. 2) (40).

However, these compounds have only the Zn-binding group together with a sidechain of penicillins, but lack the cyclic moiety and a 3-carboxylate, which is important for tight binding. One can design and synthesize cyclic, β-lactam mimicking compounds with a phosphonate, phosphinate or activated ketone group. The following compounds can be synthesized to test these possibilities:

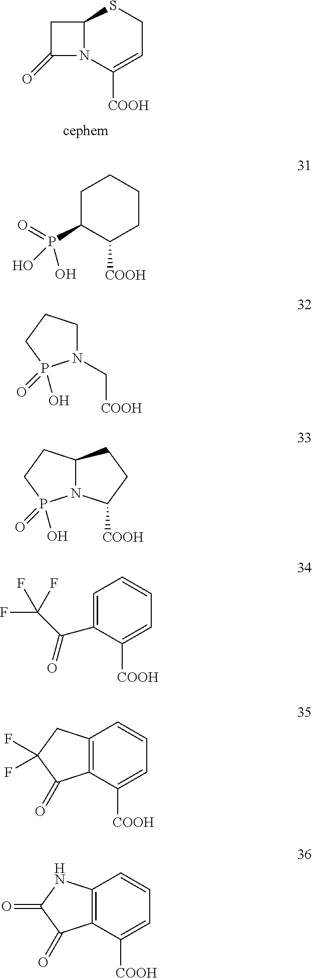

These compounds contain three elements, i.e., a Zn(II)-binding group, a carboxylate group and at least one 5- or 6-membered ring to better mimic a β-lactam. If any one of these compounds shows good activity against MBLs, one can further develop it by, for example, introducing appropriate sidechain(s) to increase the binding potency. Compounds 31-36 can be synthesized according to the following schemes (68-71), with 34 being commercially available:

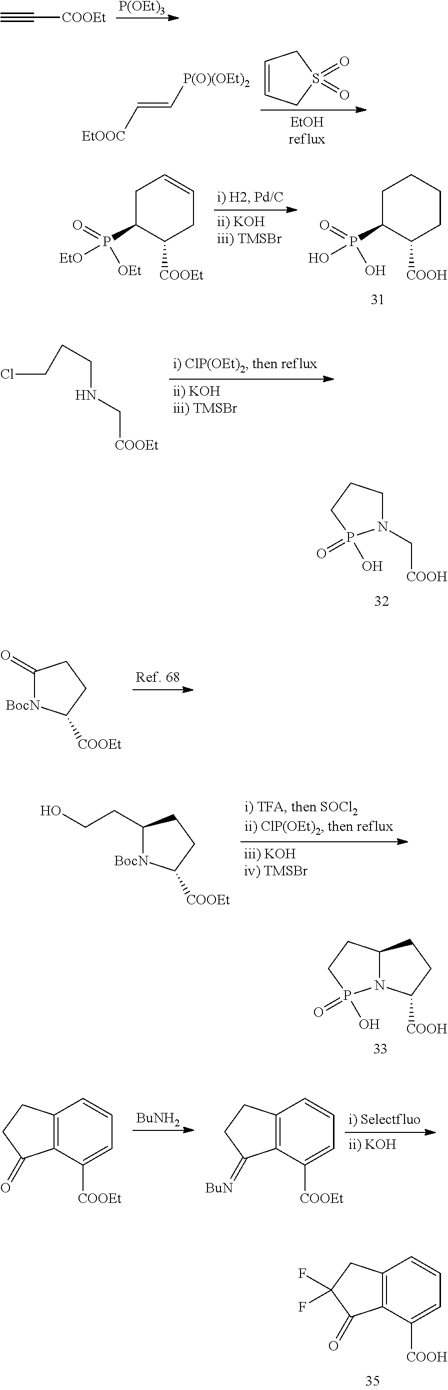

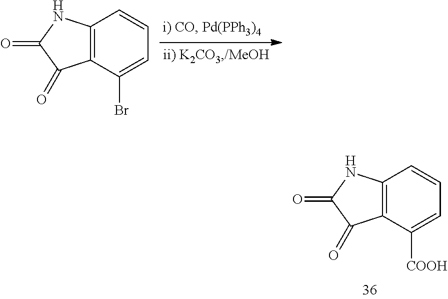

Biological activity testing. The biological activity of compounds synthesized above can be tested as described elsewhere herein.

X-ray crystallographic studies. The useful MBL inhibitors identified as described herein from the compounds shown above, x-ray crystallographic studies can be performed using IMP-1 and Bla2 with these novel inhibitors as described above.

The development of at least three series of compounds can occur: the first series is thiazolidines and their analogs, which are based on the structure of the most potent lead compound 1; the second series is based on the structure of compound 3, the second most potent lead and the third collection of potential MBL inhibitors are novel cyclic, substrate-mimicking molecules having a known Zn²⁺ binding group.

The present invention includes innovative medicinal chemistry development of inhibitors of metallo-β-lactamases, based on the inventors' recently discovered, potent lead inhibitors 1-4, as well as an x-ray crystallographic study of their structures bound to IMP-1. A coordination chemistry based approach can be combined with the development of substrate-like compounds to increase the potential of the inhibitors displaying a broad spectrum of activity. The present invention is important, given the dissemination of MBL genes, which has resulted in increased drug resistance against the last resort carbapenem antibiotics among many bacteria (e.g., Pseudomonas and Acinetobacter spp.) that already possess multi-drug resistance against many classes of antibiotics. Other studies can include the further structure based design and development of drug candidates as well as evaluation of their biological activity.

Drug design, synthesis, testing compound activities and (Q)SAR studies are performed sequentially and continuously in repeated cycles, in an effort to design, synthesize and optimize the activity. One can focus on the development of inhibitors based on compound 1, which is the most potent and promising lead inhibitor.

### Example 4

### Additional MBL Inhibitors

FIG. 8 provides additional MBL inhibitors. SYC-088 is compound 1; -031 is 2; -056 is 3 and -038 is 4.

| | |
|---|---|
| IMP-1 | Bla2 |

| Inhibitor | IC50 (μM) | KI (μM) | IC50 (μM) | KI (μM) |
|---|---|---|---|---|
| Captopril | 3 | 2.4 | 25 | |
| 031 | 34.7 | | | |
| 038 | 11.96 | | | |
| 056 | 13.94 | | | |
| 088 | 5.5 | | 376 | |
| 160 | 29.2 | | 16 | |
| 361 | | | 70 | |
| 362 | 100 | | 20 | 15 |
| 365 | 150 | | | |
| 368 | 60 | | | |
| 369 | | | 110 | |

The present invention includes embodiments wherein one or more of the compounds are provided to an individual for treatment of bacterial infection, including in instances wherein the bacteria are resistant to one or more β-lactam antibiotics.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### Patents and Patent Applications

U.S. Pat. No. 5,399,363
U.S. Pat. No. 5,466,468
U.S. Pat. No. 5,543,158
U.S. Pat. No. 5,580,579
U.S. Pat. No. 6,613,308
U.S. Pat. No. 5,629,001
U.S. Pat. No. 5,641,515
U.S. Pat. No. 5,725,871
U.S. Pat. No. 5,756,353
U.S. Pat. No. 5,780,045
U.S. Pat. No. 5,792,451
U.S. Pat. No. 5,804,212

### Publications

1. http://www.who.int/research/en/
2. Walsh, T. R.; Toleman, M. A.; Poirel, L.; Nordmann, P. Metallo-beta-lactamases: the quiet before the storm? Clin Microbiol Rev 2005, 18, 306-25.
3. Bebrone, C. Metallo-beta-lactamases (classification, activity, genetic organization, structure, zinc coordination) and their superfamily. Biochem Pharmacol 2007, 74, 1686-701.
4. Toney, J. H.; Moloughney, J. G. Metallo-beta-lactamase inhibitors: promise for the future? Curr Opin Investig Drugs 2004, 5, 823-6.
5. Walsh, T. R. The emergence and implications of metallo-beta-lactamases in Gram-negative bacteria. Clin Microbiol Infect 2005, 11 Suppl 6, 2-9.
6. Watanabe, M.; Iyobe, S.; Inoue, M.; Mitsuhashi, S. Transferable imipenem resistance in Pseudomonas aeruginosa. Antimicrob Agents Chemother 1991, 35, 147-51.
7. Minami, S.; Akama, M.; Araki, H.; Watanabe, Y.; Narita, H.; Iyobe, S.; Mitsuhashi, S. Imipenem and cephem resistant Pseudomonas aeruginosa carrying plasmids coding for class B beta-lactamase. J Antimicrob Chemother 1996, 37, 433-44.
8. Senda, K.; Arakawa, Y.; Nakashima, K.; Ito, H.; Ichiyama, S.; Shimokata, K.; Kato, N.; Ohta, M. Multifocal outbreaks of metallo-beta-lactamase-producing Pseudomonas aeruginosa resistant to broad-spectrum beta-lactams, including carbapenems. Antimicrob Agents Chemother 1996, 40, 349-53.
9. Towner, K. J.; Gee, T.; Boswell, T. An unwanted import to the UK: a carbapenem-resistant clinical isolate of Acinetobacter baumannii producing metallo-beta-lactamase. J Antimicrob Chemother 2002, 50, 1092-3.
10. Tysall, L.; Stockdale, M. W.; Chadwick, P. R.; Palepou, M. F.; Towner, K. J.; Livermore, D. M.; Woodford, N. IMP-1 carbapenemase detected in an Acinetobacter clinical isolate from the UK. J Antimicrob Chemother 2002, 49, 217-8.
11. Toney, J. H.; Fitzgerald, P. M.; Grover-Sharma, N.; Olson, S. H.; May, W. J.; Sundelof, J. G.; Vanderwall, D. E.; Cleary, K. A.; Grant, S. K.; Wu, J. K.; Kozarich, J. W.; Pompliano, D. L.; Hammond, G. G. Antibiotic sensitization using biphenyl tetrazoles as potent inhibitors of Bacteroides fragilis metallo-beta-lactamase. Chem Biol 1998, 5, 185-96.
12. Toney, J. H.; Hammond, G. G.; Fitzgerald, P. M.; Sharma, N.; Balkovec, J. M.; Rouen, G. P.; Olson, S. H.; Hammond, M. L.; Greenlee, M. L.; Gao, Y. D. Succinic acids as potent inhibitors of plasmid-borne IMP-1 metallo-beta-lactamase. J Biol Chem 2001, 276, 31913-8.
13. Leeb, M. Antibiotics: a shot in the arm. Nature 2004, 431, 892-3.
14. Dijkshoorn, L.; Nemec, A.; Seifert, H. An increasing threat in hospitals: multidrug-resistant Acinetobacter baumannii. Nat Rev Microbiol 2007, 5, 939-51.
15. Levin, A. S.; Oliveira, M. S. The challenge of multidrug resistance: the treatment of gram-negative rod infections. Shock 2008, 30 Suppl 1, 30-3.
16. McGowan, J. E., Jr. Resistance in nonfermenting gram-negative bacteria: multidrug resistance to the maximum. Am J Med 2006, 119, S29-36; discussion S62-70.
17. Rice, L. B. Challenges in identifying new antimicrobial agents effective for treating infections with Acinetobacter baumannii and Pseudomonas aeruginosa. Clin Infect Dis 2006, 43 Suppl 2, S100-5.
18. Richards, G. A. The therapeutic challenge of Gram-negative sepsis: prolonging the lifespan of a scarce resource. Clin Microbiol Infect 2005, 11 Suppl 6, 18-22.
19. Fisher, J. F.; Meroueh, S. O.; Mobashery, S. Bacterial resistance to beta-lactam antibiotics: compelling opportunism, compelling opportunity. Chem Rev 2005, 105, 395-424.
20. Crowder, M. W.; Spencer, J.; Vila, A. J. Metallo-beta-lactamases: novel weaponry for antibiotic resistance in bacteria. Acc Chem Res 2006, 39, 721-8.
21. Wang, Z.; Fast, W.; Valentine, A. M.; Benkovic, S. J. Metallo-beta-lactamase: structure and mechanism. Curr Opin Chem Biol 1999, 3, 614-22.
22. Singh, G. S. Beta-lactams in the new millennium. Part-II: cephems, oxacephems, penams and sulbactam. Mini Rev Med Chem 2004, 4, 93-109.
23. Sabath, L. D.; Abraham, E. P. Zinc as a cofactor for cephalosporinase from Bacillus cereus 569. Biochem J 1966, 98, 11C-3C.
24. Senda, K.; Arakawa, Y.; Ichiyama, S.; Nakashima, K.; Ito, H.; Ohsuka, S.; Shimokata, K.; Kato, N.; Ohta, M. PCR detection of metallo-beta-lactamase gene (blaIMP) in gram-negative rods resistant to broad-spectrum beta-lactams. J Clin Microbiol 1996, 34, 2909-13.
25. Shibata, N.; Doi, Y.; Yamane, K.; Yagi, T.; Kurokawa, H.; Shibayama, K.; Kato, H.; Kai, K.; Arakawa, Y. PCR typing of genetic determinants for metallo-beta-lactamases and integrases carried by gram-negative bacteria isolated in Japan, with focus on the class 3 integron. J Clin Microbiol 2003, 41, 5407-13.
26. Osano, E.; Arakawa, Y.; Wacharotayankun, R.; Ohta, M.; Horii, T.; Ito, H.; Yoshimura, F.; Kato, N. Molecular characterization of an enterobacterial metallo beta-lactamase found in a clinical isolate of Serratia marcescens that shows imipenem resistance. Antimicrob Agents Chemother 1994, 38, 71-8.
27. Arakawa, Y.; Murakami, M.; Suzuki, K.; Ito, H.; Wacharotayankun, R.; Ohsuka, S.; Kato, N.; Ohta, M. A novel integron-like element carrying the metallo-beta-lactamase gene blaIMP. Antimicrob Agents Chemother 1995, 39, 1612-5.
28. Lee, K.; Lee, W. G.; Uh, Y.; Ha, G. Y.; Cho, J.; Chong, Y. VIM- and IMP-type metallo-beta-lactamase-producing Pseudomonas spp. and Acinetobacter spp. in Korean hospitals. Emerg Infect Dis 2003, 9, 868-71.
29. Lauretti, L.; Riccio, M. L.; Mazzariol, A.; Cornaglia, G.; Amicosante, G.; Fontana, R.; Rossolini, G. M. Cloning and characterization of blaVIM, a new integron-borne metallo-beta-lactamase gene from a Pseudomonas aeruginosa clinical isolate. Antimicrob Agents Chemother 1999, 43, 1584-90.
30. Poirel, L.; Collet, L.; Nordmann, P. Carbapenem-hydrolyzing metallo-beta-lactamase from a nosocomial isolate of Pseudomonas aeruginosa in France. Emerg Infect Dis 2000, 6, 84-5.
31. Poirel, L.; Naas, T.; Nicolas, D.; Collet, L.; Bellais, S.; Cavallo, J. D.; Nordmann, P. Characterization of VIM-2, a carbapenem-hydrolyzing metallo-beta-lactamase and its plasmid- and integron-borne gene from a Pseudomonas aeruginosa clinical isolate in France. Antimicrob Agents Chemother 2000, 44, 891-7.
32. Yan, J. J.; Hsuch, P. R.; Ko, W. C.; Luh, K. T.; Tsai, S. H.; Wu, H. M.; Wu, J. J. Metallo-beta-lactamases in clinical Pseudomonas isolates in Taiwan and identification of VIM-3, a novel variant of the VIM-2 enzyme. Antimicrob Agents Chemother 2001, 45, 2224-8.
33. Pournaras, S.; Tsakris, A.; Maniati, M.; Tzouvelekis, L. S.; Maniatis, A. N. Novel variant (bla(VIM-4)) of the metallo-beta-lactamase gene bla(VIM-1) in a clinical strain of Pseudomonas aeruginosa. Antimicrob Agents Chemother 2002, 46, 4026-8.

34. Koh, T. H.; Wang, G. C.; 5 ng, L. H. IMP-1 and a novel metallo-beta-lactamase, VIM-6, in fluorescent pseudomonads isolated in Singapore. Antimicrob Agents Chemother 2004, 48, 2334-6.
35. Castanheira, M.; Toleman, M. A.; Jones, R. N.; Schmidt, F. J.; Walsh, T. R. Molecular characterization of a beta-lactamase gene, blaGIM-1, encoding a new subclass of metallo-beta-lactamase. Antimicrob Agents Chemother 2004, 48, 4654-61.
36. Garcia-Saez, I.; Hopkins, J.; Papamicael, C.; Franceschini, N.; Amicosante, G.; Rossolini, G. M.; Galleni, M.; Frere, J. M.; Dideberg, O. The 1.5-A structure of Chryseobacterium meningosepticum zinc beta-lactamase in complex with the inhibitor, D-captopril. J Biol Chem 2003, 278, 23868-73.
37. Heinz, U.; Bauer, R.; Wommer, S.; Meyer-Klaucke, W.; Papamichaels, C.; Bateson, J.; Adolph, H. W. Coordination geometries of metal ions in d- or l-captopril-inhibited metallo-beta-lactamases. J Biol Chem 2003, 278, 20659-66.
38. Buynak, J. D.; Chen, H.; Vogeti, L.; Gadhachanda, V. R.; Buchanan, C. A.; Palzkill, T.; Shaw, R. W.; Spencer, J.; Walsh, T. R. Penicillin-derived inhibitors that simultaneously target both metallo- and serine-beta-lactamases. Bioorg Med Chem Lett 2004, 14, 1299-304.
39. Goto, M.; Takahashi, T.; Yamashita, F.; Koreeda, A.; Mori, H.; Ohta, M.; Arakawa, Y. Inhibition of the metallo-beta-lactamase produced from Serratia marcescens by thiol compounds. Biol Pharm Bull 1997, 20, 1136-40.
40. Walter, M. W.; Felici, A.; Galleni, M.; Soto, R. P.; Adlington, R. M.; Baldwin, J. E.; Frere, J.-M.; Gololobov, M.; Schofield, C. J. Trifluoromethyl alcohol and ketone inhibitors of metallo-[beta]-lactamases. Bioorganic & Medicinal Chemistry Letters 1996, 6, 2455-2458.
41. Concha, N. O.; Janson, C. A.; Rowling, P.; Pearson, S.; Cheever, C. A.; Clarke, B. P.; Lewis, C.; Galleni, M.; Frere, J. M.; Payne, D. J.; Bateson, J. H.; Abdel-Meguid, S. S. Crystal structure of the IMP-1 metallo beta-lactamase from Pseudomonas aeruginosa and its complex with a mercaptocarboxylate inhibitor: binding determinants of a potent, broad-spectrum inhibitor. Biochemistry 2000, 39, 4288-98.
42. Scrofani, S. D.; Chung, J.; Huntley, J. J.; Benkovic, S. J.; Wright, P. E.; Dyson, H. J. NMR characterization of the metallo-beta-lactamase from Bacteroides fragilis and its interaction with a tight-binding inhibitor: role of an active-site loop. Biochemistry 1999, 38, 14507-14.
43. Hudock, M. P.; Sanz-Rodriguez, C. E.; Song, Y.; Chan, J. M.; Zhang, Y.; Odeh, S.; Kosztowski, T.; Leon-Rossell, A.; Concepcion, J. L.; Yardley, V.; Croft, S. L.; Urbina, J. A.; Oldfield, E. Inhibition of Trypanosoma cruzi hexokinase by bisphosphonates. J Med Chem 2006, 49, 215-23.
44. Kotsikorou, E.; Song, Y.; Chan, J. M.; Faelens, S.; Tovian, Z.; Broderick, E.; Bakalara, N.; Docampo, R.; Oldfield, E. Bisphosphonate inhibition of the exopolyphosphatase activity of the Trypanosoma brucei soluble vacuolar pyrophosphatase. J Med Chem 2005, 48, 6128-39.
45. Song, Y.; Chan, J. M.; Tovian, Z.; Secrest, A.; Nagy, E.; Krysiak, K.; Bergan, K.; Parniak, M. A.; Oldfield, E. Bisphosphonate inhibitors of ATP-mediated HIV-1 reverse transcriptase catalyzed excision of chain-terminating 3′-azido, 3′-deoxythymidine: a QSAR investigation. Bioorg Med Chem 2008, 16, 8959-67.
46. Chen, C. K. M.; Hudock, M. P.; Zhang, Y.; Guo, R. T.; Cao, R.; No, J. H.; Liang, P. H.; Ko, T. P.; Chang, T. H.; Chang, S. C.; Song, Y.; Axelson, J.; Kumar, A.; Wang, A. H.; Oldfield, E. Inhibition of geranylgeranyl diphosphate synthase by bisphosphonates: a crystallographic and computational investigation. J Med Chem 2008, 51, 5594-607.
47. Guo, R. T.; Cao, R.; Liang, P. H.; Ko, T. P.; Chang, T. H.; Hudock, M. P.; Jeng, W. Y.; Chen, C. K.; Zhang, Y.; Song, Y.; Kuo, C. J.; Yin, F.; Oldfield, E.; Wang, A. H. Bisphosphonates target multiple sites in both cis- and trans-prenyltransferases. Proc Natl Acad Sci USA 2007, 104, 10022-7.
48. Zhang, Y.; Cao, R.; Yin, F.; Hudock, M. P.; Guo, R. T.; Krysiak, K.; Mukherjee, S.; Gao, Y. G.; Robinson, H.; Song, Y.; No, J. H.; Bergan, K.; Leon, A.; Cass, L.; Goddard, A.; Chang, T. K.; Lin, F. Y.; Van Beek, E.; Papapoulos, S.; Wang, A. H.; Kubo, T.; Ochi, M.; Mukkamala, D.; Oldfield, E. Lipophilic bisphosphonates as dual farnesyl/geranylgeranyl diphosphate synthase inhibitors: an X-ray and NMR investigation. J Am Chem Soc 2009, 131, 5153-62.
49. Liu, C. I.; Liu, G. Y.; Song, Y.; Yin, F.; Hensler, M. E.; Jeng, W. Y.; Nizet, V.; Wang, A. H.; Oldfield, E. A cholesterol biosynthesis inhibitor blocks Staphylococcus aureus virulence. Science 2008, 319, 1391-4.
50. Song, Y.; Lin, F. Y.; Yin, F.; Hensler, M.; Rodrígues-Poveda, C. A.; Mukkamala, D.; Cao, R.; Wang, H.; Morita, C. T.; González-Pacanowska, D.; Nizet, V.; Oldfield, E. Phosphonosulfonates Are Potent, Selective Inhibitors of Dehydrosqualene Synthase and Staphyloxanthin Biosynthesis in Staphylococcus aureus. J Med Chem 2008, 52, 976-88.
51. Song, Y.; Liu, C. I.; Lin, F. Y.; No, J. H.; Hensler, M.; Liu, Y. L.; Jeng, W. Y.; Low, J.; Liu, G. Y.; Nizet, V.; Wang, A. H.; Oldfield, E. Inhibition of Staphyloxanthin Virulence Factor Biosynthesis in Staphylococcus aureus: In Vitro, in Vivo, and Crystallographic Results. J Med Chem 2009, in press.
52. Clauditz, A.; Resch, A.; Wieland, K. P.; Peschel, A.; Gotz, F. Staphyloxanthin plays a role in the fitness of Staphylococcus aureus and its ability to cope with oxidative stress. Infect Immun 2006, 74, 4950-3.
53. Liu, G. Y.; Essex, A.; Buchanan, J. T.; Datta, V.; Hoffman, H. M.; Bastian, J. F.; Fierer, J.; Nizet, V. Staphylococcus aureus golden pigment impairs neutrophil killing and promotes virulence through its antioxidant activity. J Exp Med 2005, 202, 209-15.
54. Deng, L.; Sundriyal, S.; Rubio, V.; Shi, Z.; Song, Y. A Coordination Chemistry Based Approach to Lipophilic Inhibitors of 1-Deoxy-D-xylulose-5-phosphate Reductoisomerase. J Med Chem 2009, in press.
55. Walsh, T. R. Clinically significant carbapenemases: An update. Curr. Opin. Infect. Dis. 2008, 21, 367-371.
56. Concha, N. O.; Janson, C. A.; Rowling, P.; Pearson, S.; Cheever, C. A.; Clarke, B. P.; Lewis, C.; Galleni, M.; Frere, J.-M.; Payne, D. J.; Bateson, J. H.; Abdel-Meguid, S. S. Crystal structure of the IMP-1 metallo b-lactamase from Pseudomonas aeruginosa and its complex with a mercaptocarboxylate inhibitor: binding determinants of a potent, broad-spectrum inhibitor. Biochemistry 2000, 39, 4288-4298.
57. Materon, I. C.; Beharry, Z.; Huang, W.; Perez, C.; Palzkill, T. Analysis of the context dependent sequence requirements of active site residues in the metallo-beta-lactamase IMP-1. J. Mol. Biol. 2004, 344, 653-663.
58. Materon, I. C.; Palzkill, T. Identification of residues critical for metallo-b-lactamase function by codon randomization and selection. Protein Sci. 2001, 10, 2556-2565.
59. Materon, I. C.; Queenan, A. M.; Koehler, T. M.; Bush, K.; Palzkill, T. Biochemical characterization of b-lactamase enzymes Bla1 and Bla2 from Bacillus anthracis. Antimicrob. Agents Chemother. 2003, 47, 2040-2042.
60. Spencer, J.; Read, J.; Sessions, R. B.; Howell, S.; Blackburn, G. M.; Gamblin, S. J. Antibiotic recognition by binuclear metallo-beta-lactamases revealed by X-ray crystallography. J Am Chem Soc 2005, 127, 14439-44.
61. Garau, G.; Bebrone, C.; Anne, C.; Galleni, M.; Frere, J. M.; Dideberg, O. A metallo-beta-lactamase enzyme in action: crystal structures of the monozinc carbapenemase CphA and its complex with biapenem. J Mol Biol 2005, 345, 785-95.
62. Klebe, G.; Abraham, U.; Mietzner, T. Molecular similarity indices in a comparative analysis (CoMSIA) of drug molecules to correlate and predict their biological activity. J Med Chem 1994, 37, 4130-46.
63. Cramer, R. D., 3rd; Patterson, D. E.; Bunce, J. D. Recent advances in comparative molecular field analysis (CoMFA). Prog Clin Biol Res 1989, 291, 161-5.
64. Copeland, R. A. Enzymes: A practical introduction to structure, mechanism, and data analysis. 2nd ed.; Wiley-VCH: New York, 2000.
65. Kurosaki, H.; Yamaguchi, Y.; Higashi, T.; Soga, K.; Matsueda, S.; Yumoto, H.; Misumi, S.; Yamagata, Y.; Arakawa, Y.; Goto, M. Irreversible inhibition of metallo-beta-lactamase (IMP-1) by 3-(3-mercaptopropionylsulfanyl)propionic acid pentafluorophenyl ester. Angew Chem Int Ed Engl 2005, 44, 3861-4.
66. Kurosaki, H.; Yamaguchi, Y.; Yasuzawa, H.; Jin, W.; Yamagata, Y.; Arakawa, Y. Probing, inhibition, and crystallographic characterization of metallo-beta-lactamase (IMP-1) with fluorescent agents containing dansyl and thiol groups. Chem Med Chem 2006, 1, 969-72.
67. Horsfall, L. E.; Garau, G.; Lienard, B. M.; Dideberg, O.; Schofield, C. J.; Frere, J. M.; Galleni, M. Competitive inhibitors of the CphA metallo-beta-lactamase from Aeromonas hydrophila. Antimicrob Agents Chemother 2007, 51, 2136-42.
68. Mulzer, J.; Schulzchen, F.; Bats, J.-W. Rigid Depeptide Mimetics. Stereocontrolled synthesis of all eight stereoisomers of 2-oxo-3-(N-Cbz-amino)-1-azabicyclo[4.3.0]nonane-9-carboxylic acid ester. Tetrahedron 2000, 56, 4289-98.
69. Aladzheva, I. M.; Bykhovskaya, O. V.; Petrovskii, P. V.; Lyssenko, K. A.; Antipin, M. Y.; Mastryukova, T. A. N-(3-Phosphonopropyl)-substituted alpha-amino acids and their phosphine oxide analogs. Russian Chemical Bulletin, International Edition 2005, 54, 2635-2641.
70. Bessis, A. S.; Vadesne, G.; Bourrat, E.; Bertho, G.; Pin, J. P.; Acher, F. C. 3-Carboxy-4-phosphonocyclopentane amino acids: New metabotropic glutamate receptor ligands. Amino Acids 2003, 303-310.
71. Webber, S. E.; Tikhe, J.; Worland, S. T.; Fuhrman, S. A.; Hendrickson, T. F.; Matthews, D. A.; Love, R. A.; Patick, A. K.; Meador, J. W.; Ferre, R. A.; Brown, E. L.; DeLisle, D. M.; Ford, C. E.; Binford, S. L. Design, synthesis, and evaluation of nonpeptidic inhibitors of human rhinovirus 3C protease. J Med Chem 1996, 39, 5072-82.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A metallo-β-lactamase inhibitor having the formula: 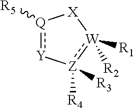
wherein
X is S, SO₂, or NH
Y is N or NH
R₁ is independently selected from hydrogen, unsubstituted alkyl_{(C1-12)}, unsubstituted aryl, substituted alkyl_{(C1-12)}, substituted aryl, heteroaryl, heterocycle or acyl;
R₂ is independently selected from hydrogen, unsubstituted alkyl_{(C1-12)}, unsubstituted aryl, substituted alkyl_{(C1-12)}, substituted aryl, heteroaryl, heterocycle or acyl;
R₃ is independently selected from hydrogen, unsubstituted alkyl_{(C1-12)}, unsubstituted aryl, substituted alkyl_{(C1-12)}, substituted aryl, heteroaryl, heterocycle or acyl;
R₄ is independently selected from hydrogen, unsubstituted alkyl_{(C1-12)}, unsubstituted aryl, substituted alkyl_{(C1-12)}, substituted aryl, heteroaryl, heterocycle or acyl;
R₅ is independently selected from unsubstituted alkyl_{(C1-12)}, unsubstituted aryl, substituted alkyl_{(C1-12)}, substituted aryl, heteroaryl, heterocycle or acyl;
Q is C or CH;
W is C; and,
Z is C.

2. The metallo-β-lactamase inhibitor of claim 1 wherein X is S.

3. The metallo-β-lactamase inhibitor of claim 2 wherein Y is N and Q is C.

4. The metallo-β-lactamase inhibitor of claim 3 wherein R₅ is

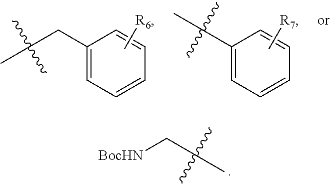

5. The metallo-β-lactamase inhibitor of claim 4 wherein R₆ is hydrogen, halogen, alkyl_{(c≦5)}, hydroxy, —NH₂, —NO₂, —CO₂H, —CO₂CH₃, —CN, —SH, —OCH₃, —OCH₂CH₃, —C(O)CH₃, —N(CH₃)₂, —C(O)NH₂, —NHAc, or —NHBoc.

6. The metallo-β-lactamase inhibitor of claim 4 wherein R₇ is hydrogen, halogen, alkyl_{(c≦5)}, hydroxy, —NH₂, —NO₂, —CO₂H, —CO₂CH₃, —CN, —SH, —OCH₃, —OCH₂CH₃, —C(O)CH₃, —N(CH₃)₂, —C(O)NH₂, —NHAc, or —NHBoc.

7. The metallo-β-lactamase inhibitor of claim 3 wherein R₁ is acyl.

8. The metallo-β-lactamase inhibitor of claim 7 wherein R₁ is CO₂R₈.

9. The metallo-β-lactamase inhibitor of claim 8 wherein R₈ is alkyl_{(c≦5)}.

10. The metallo-β-lactamase inhibitor of claim 3 wherein R₂ is acyl.

11. The metallo-β-lactamase inhibitor of claim 10 wherein R₂ is CO₂R₉.

12. The metallo-β-lactamase inhibitor of claim 11 wherein R₉ is alkyl_{(c≦5)}.

13. The metallo-β-lactamase inhibitor of claim 3 wherein R₃ is acyl.

14. The metallo-β-lactamase inhibitor of claim 13 wherein R₃ is CO₂H.

15. The metallo-β-lactamase inhibitor of claim 3 wherein R₄ is acyl.

16. The metallo-β-lactamase inhibitor of claim 15 wherein R₄ is CO₂H.

17. The metallo-β-lactamase inhibitor of claim 1 wherein the metallo-β-lactamase inhibitor has a formula selected from the group consisting of:

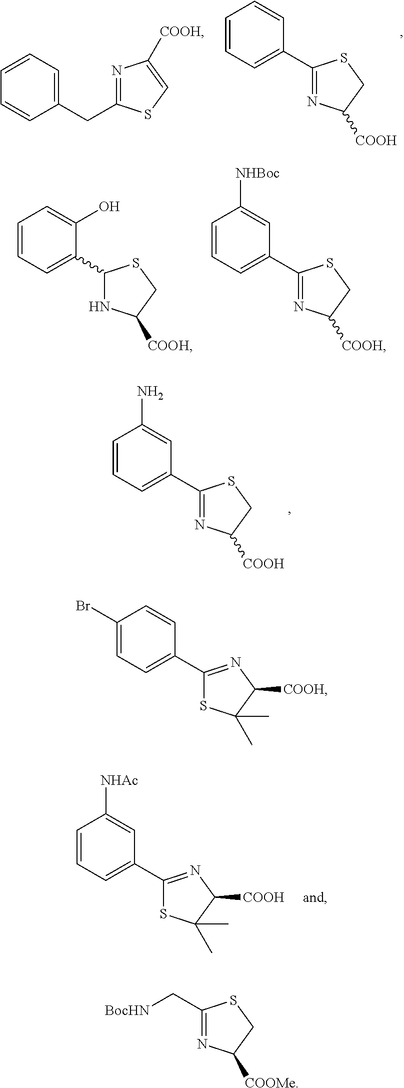

18. A method for inhibiting a metallo-β-lactamase comprising the step of:
administering to the metallo-β-lactamase a composition having the formula: 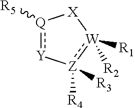
wherein
X is S, SO₂, or NH
Y is N or NH
R₁ is independently selected from hydrogen, unsubstituted alkyl_{(C1-12)}, unsubstituted aryl, substituted alkyl_{(C1-12)}, substituted aryl, heteroaryl, heterocycle or acyl;
R₂ is independently selected from hydrogen, unsubstituted alkyl_{(C1-12)}, unsubstituted aryl, substituted alkyl_{(C1-12)}, substituted aryl, heteroaryl, heterocycle or acyl;
R₃ is independently selected from hydrogen, unsubstituted alkyl_{(C1-12)}, unsubstituted aryl, substituted alkyl_{(C1-12)}, substituted aryl, heteroaryl, heterocycle or acyl;
R₄ is independently selected from hydrogen, unsubstituted alkyl_{(C1-12)}, unsubstituted aryl, substituted alkyl_{(C1-12)}, substituted aryl, heteroaryl, heterocycle or acyl;
R₅ is independently selected from unsubstituted alkyl_{(C1-12)}, unsubstituted aryl, substituted alkyl_{(C1-12)}, substituted aryl, heteroaryl, heterocycle or acyl;
Q is C or CH;
W is C; and,
Z is C.

19. The metallo-β-lactamase inhibitor of claim 18 wherein X is S.

20. The metallo-β-lactamase inhibitor of claim 19 wherein Y is N and Q is C.

21. The metallo-β-lactamase inhibitor of claim 20 wherein R₅ is

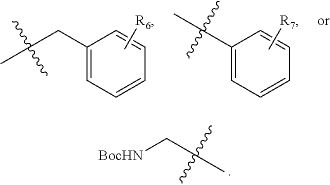

22. The metallo-β-lactamase inhibitor of claim 21 wherein R₆ is hydrogen, halogen, alkyl_{(c≦5)}, hydroxy, —NH₂, —NO₂, —CO₂H, —CO₂CH₃, —CN, —SH, —OCH₃, —OCH₂CH₃, —C(O)CH₃, —N(CH₃)₂, —C(O)NH₂, —NHAc, or —NHBoc.

23. The metallo-β-lactamase inhibitor of claim 21 wherein R₇ is hydrogen, halogen, alkyl_{(c≦5)}, hydroxy, —NH₂, —NO₂, —CO₂H, —CO₂CH₃, —CN, —SH, —OCH₃, —OCH₂CH₃, —C(O)CH₃, —N(CH₃)₂, —C(O)NH₂, —NHAc, or —NHBoc.

24. The metallo-β-lactamase inhibitor of claim 20 wherein R₁ is acyl.

25. The metallo-β-lactamase inhibitor of claim 24 wherein R₁ is CO₂R₈.

26. The metallo-β-lactamase inhibitor of claim 25 wherein R₈ is alkyl_{(c≦5)}.

27. The metallo-β-lactamase inhibitor of claim 20 wherein R₂ is acyl.

28. The metallo-β-lactamase inhibitor of claim 27 wherein R₂ is CO₂R₉.

29. The metallo-β-lactamase inhibitor of claim 28 wherein R₉ is alkyl_{(c≦5)}.

30. The metallo-β-lactamase inhibitor of claim 20 wherein R₃ is acyl.

31. The metallo-β-lactamase inhibitor of claim 30 wherein R₃ is CO₂H.

32. The metallo-β-lactamase inhibitor of claim 20 wherein R₄ is acyl.

33. The metallo-β-lactamase inhibitor of claim 32 wherein R₄ is CO₂H.

34. The metallo-β-lactamase inhibitor of claim 18 wherein the metallo-β-lactamase inhibitor has a formula selected from the group consisting of:

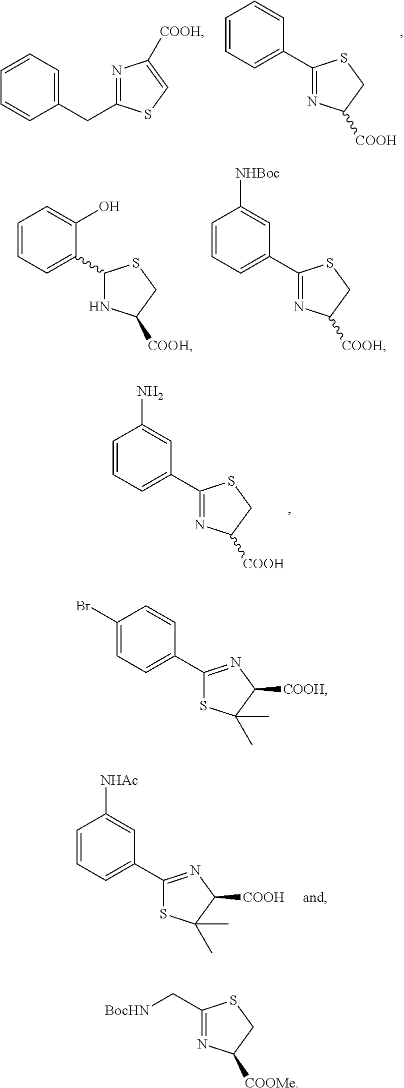

36. The method of claim 18, wherein the metallo-β-lactamase is in or is secreted from a bacteria in an individual.

37. A method of treating and/or preventing a bacterial infection in an individual, comprising the step of providing a therapeutically effective amount of an inhibitor of claim 1 to the individual, wherein the bacteria produces at least one metallo-β-lactamase.

38. The method of claim 37, wherein the method further comprises providing to the individual a β-lactam antibiotic.

39. The method of claim 38, wherein the inhibitor and the β-lactam antibiotic are provided to the individual at the same time.

40. The method of claim 39, wherein the inhibitor and β-lactam antibiotic are provided to the individual in the same formulation.

41. The method of claim 40, wherein the formulation is in the form of a pill, liquid, injection, aerosol, or cream.

42. The method of claim 39, wherein the inhibitor and the β-lactam antibiotic are provided to the individual in different formulations.

43. The method of claim 38, wherein the inhibitor and the β-lactam antibiotic are provided to the individual at different times.

44. The method of claim 37, wherein the method further comprises providing an additional therapy to the individual.

45. The method of claim 44, wherein the additional therapy comprises another antibiotic, analgesic, cough suppressant, or a combination thereof.

46. The method of claim 37, wherein the bacteria are resistant to one or more antibiotics.

47. The method of claim 38, wherein the bacteria are resistant to the β-lactam antibiotic.
